(19) United States
(12) Patent Application Publication
(10) Pub. No.: US 2022/0025430 A1
(43) Pub. Date: Jan. 27, 2022

(54) SEQUENCE BASED IMAGING

(71) Applicant: Colin THOM, Stanford, CA (US)

(72) Inventor: Colin THOM, Stanford, CA (US)

(21) Appl. No.: 17/450,347

(22) Filed: Oct. 8, 2021

Related U.S. Application Data
(63) Continuation of application No. PCT/US2020/031892, filed on May 7, 2020.
(60) Provisional application No. 63/016,582, filed on Apr. 28, 2020.
(60) Provisional application No. 62/844,270, filed on May 7, 2019.

(51) Int. Cl. C12Q 1/682

(52) U.S. Cl. CPC C12Q 1/682

(57) ABSTRACT

Described herein are a variety of method, reagents and kits for determining and/or representing the spatial relationships of oligonucleotide probes (or probe targets) in a biological sample. Such representation may take the form of encoding spatial relationships in oligonucleotide sequences of reaction products, sequencing of spatially encoded reaction products, and analyzing or visualizing spatial relationships based on the spatially encoded sequences.

### CROSS-REFERENCE TO RELATED APPLICATIONS

This application is a continuation of International Patent Application No. PCT/US2020/031892 filed May 7, 2020, which claims benefit of priority to U.S. Provisional Application Nos. 63/016,582 filed Apr. 28, 2020, and 62/844,270 filed May 7, 2019, the content of each of which is incorporated herein by reference in its entirety.

### BACKGROUND OF THE INVENTION

Imaging technologies struggle to maximize both spatial resolution and plexity of biological targets (e.g., the number of different protein, DNA and/or RNA targets detected). For example, electron microscopy can image a biological sample at high resolution (on the scale of tens of nanometers) but can only detect one target, such as a protein bound by an antibody tagged with a dense nanoparticle. Fluorescence microscopy can detect several targets, but has worse resolution compared to electron microscopy (on the micrometer scale). Emerging technologies that allow dozens of protein targets to be imaged simultaneously often struggle to achieve similar resolution to fluorescence microscopy (e.g., cellular or subcellular resolution), much less high resolution. Sequencing, as opposed to other methods of detection such as fluorescence microscopy, allows for thousands of unique molecules (oligonucleotide sequences) to be identified, but fails to achieve spatial resolution similar to that of fluorescent microscopy. Such spatial limitations of sequencing-based imaging approaches are in part due to the physical need to spatially organize barcoded oligonucleotide probes prior to applying them to a sample, or harvesting oligonucleotide probes (or their reaction products) from known regions of a sample and associating harvested oligonucleotides with regional barcodes.

### BRIEF SUMMARY OF THE INVENTION

Described herein are compositions, kits, samples and methods for representing the spatial relationships between targets, oligonucleotide probes and/or cells in a biological sample. Spatial relationships may be represented by encoding at different levels including incorporation of spatially indicative variable sequences and/or spatial barcodes in reaction products, sequencing of the reaction products, and/or rendering of spatial relationships in a user interface base on the sequences of the reaction products.

A method of representing the spatial relationships in a sample may include contacting a cellular sample, such as a tissue section, with oligonucleotide probes and forming a plurality of reaction products from the oligonucleotide probes. A first plurality of the oligonucleotide probes may each comprise a variable sequence. A second plurality of the oligonucleotide probes may be target-specific probes that each comprise a target barcode sequences that identifies a target that is specifically bound by the target-specific probe, and may include an affinity reagent, such as an antibody, that specifically binds the target. The first and second plurality of probes may be the same population, a different population, or a partially overlapping population (e.g., such that some probes include both a variable sequence and a target barcode sequence.

Forming a plurality of reaction products from the oligonucleotide probes may be in the presence of a polymerase, such that probes extend off other probes to form reaction products. The reaction products may incorporate a variable sequence from at least three oligonucleotide probes, and may further include at least one target barcode sequence. Such reaction products may be formed while immobilized in the cellular sample.

Forming the plurality of the reaction products may be by successive extension, e.g., such that a first extension of a first extending oligonucleotide probe along a first template oligonucleotide probe, wherein the first extension terminates in a sequence that hybridizes to, and extends along, a second template oligonucleotide probe (or its reaction product). The first and/or second template oligonucleotide probes may be target-specific probes (such as a target specific probe comprising an oligonucleotide conjugated to an affinity reagent). Alternatively or in addition, the first extending oligonucleotide probe may be a target-specific probe. The extending probe may terminate in a 3′ sequence that hybridizes to a template probe or reaction sequence and, after extension, may terminate in another 3′ sequence that can again hybridized to and extend along another template probe or reaction product thereof.

The reaction products may be formed by unidirectional extension as described further herein, such that the first and/or second template oligonucleotide probes do not extend. Alternatively, the reaction products may be formed by bidirectional extension such that at least some of the template probes also extend along the first extending oligonucleotide probe.

In certain aspects, the plurality of the reaction products of step are immobilized in the cellular sample through binding of an affinity reagent (such as binding of an antibody to analyte in the cellular sample). Alternatively or in addition, a plurality of the reaction products of step may be immobilized in the cellular sample through covalent binding to functional groups presented by molecules endogenous to the sample. In certain aspects, a plurality of the reaction products of step b) are bound through hybridization to a plurality of oligonucleotide probes that are themselves bound to the cellular sample (such as by at least one of a covalent bond or through binding of an affinity reagent).

The method may further include applying a spatial barcode to the sample as described herein, wherein a plurality of the reaction products comprise the spatial barcode. The method may further comprising disaggregation of cells from the cellular sample, isolation of individual cells with a cell barcode, lysis, and indexing (incorporation) of cell barcode into reaction products of individual disaggregated cells with a cell barcode. Alternatively or in addition, fragments of tissue (as opposed to single cells) may be dissociated and processed in this way.

Association of spatial barcodes applied to a sample with cell barcodes after dissociation is an example of nested spatial barcode concept described herein, in which on spatial barcode (in this case, a cell barcode) is used to relate the proximity of other spatial barcodes (the spatial barcodes applied to the sample prior to dissociation).

In certain aspects, the majority of oligonucleotide probes encoded in a reaction product of the plurality of reaction products are within 100 nm of the majority of other oligonucleotide probes in the same reaction product. This may be due to the reaction product forming from proximal bound probes (probes close enough that the probe or its reaction product can reach and hybridized to other probes that are incorporated into the reaction product). In certain aspects, some but not all of the probes incorporated into the reaction product may be free (unbound), such as spatial barcode probes discussed herein.

Any of the above methods may further include sequencing the plurality of reaction products. As described further herein, the co-occurrence of variable sequences in the same reaction product and/or spatial barcodes shared across reaction products, may be used to identify probes that were in proximity in the cellular sample. As such, a relativistic spatial relationships of the probes (e.g., or probe targets) may be determined. The method may further comprise rendering the spatial relationships of the oligonucleotide probes or their targets, based on sequences of the reaction product. Such rendering may use software to display the localization of cells in the sample, identify cell types (e.g., expression profiles), as described further herein. Such software is within the scope of the subject application.

In certain aspects, a method of representing spatial relationships in a sample may include contacting a cellular sample with oligonucleotide probes, wherein a plurality of the oligonucleotide probes comprise variable sequences, and forming reaction products from the oligonucleotide probes, wherein a plurality of the reaction products comprise variable sequences from at least three oligonucleotide probes and are formed while immobilized in the cellular sample. The method may further include sequencing the reaction products of step b) and optionally rendering the spatial relationships of probes as described herein.
A method of representing the spatial relationships in a sample may include contacting a cellular sample with oligonucleotide probes, wherein a plurality of the oligonucleotide probes are target-specific oligonucleotide probes that comprise a target barcode sequence, and forming reaction products from the oligonucleotide probes, wherein a plurality of the reaction products comprise target barcode sequences from at least three target-specific oligonucleotide probes. The reaction products may include a plurality of variable sequences from different oligonucleotide probes. The method may further include sequencing the reaction products of step b) and optionally rendering the spatial relationships of probes as described herein.

Aspects include a set of oligonucleotide probes for identifying spatial relationships within a cellular sample, wherein a plurality of the oligonucleotide probes comprise variable sequences, a plurality of the oligonucleotide probes are target-specific oligonucleotide probes each comprising a target barcode sequences that identifies a target that is specifically bound by the target-specific oligonucleotide probe. The oligonucleotide probes of the set may react to form reaction products in the sample by successive extension, wherein a plurality of the reaction products comprise variable sequences from at least three oligonucleotide probes and at least one target barcode sequence, and wherein the plurality of reaction products are formed while immobilized in the cellular sample. The set may be part of a kit, and may include other kit components as described herein. In certain aspects, a composition or kit may include a set of probes that can be added to a sample and reacted to provide reaction products whose sequences together identify the relative spatial relationships between probes in the sample.

One or more of the probes in the set may be target-specific probes (e.g., oligonucleotides) that specifically bind to one or more targets in the sample. For example, probes may bind a nucleic acid target (e.g., RNA) through hybridization. Such RNA target specific probes may be extendable along the target (e.g., from a S′ end, such as a target specific or 3′ poly-T tail). Alternatively or in addition, probes may bind to targets through an intermediate affinity reagent, such as binding to protein through an antibody intermediate.

The set of oligonucleotides may further include reference oligonucleotide probes which are distributed throughout the sample as described herein. The reference probes may be functionalized to bind non-specifically throughout the sample, such as through a covalent interaction. Binding may be by covalent binding to common functional groups presented by a variety of molecules in the sample, or binding to a gel (such as an expansion gel). For example, reference probes functionalized with lysine (e.g., poly-lysine) may be distributed through the sample by diffusion, and bound to the sample during a fixation step (e.g., by formaldehyde).

The oligonucleotides in the set may be distributed throughout the sample (e.g., may form a 2D or 3D lawn across the sample) such that distant oligonucleotides may react with the same intermediate oligonucleotides but not each other.

In certain aspects, oligonucleotide probes in the set may comprise at least one reactive sequence such as a hybridization sequence (e.g., the reverse complement of another hybridization sequence on the same probe, or on another probe in the set), digestion and/or ligation sequences, transposable sequences, or another reactive sequence known in the art. Reactive sequences of oligonucleotide probes in the set may allow concatemerization of a plurality of probes (e.g., 3, 4, 5, 8, 10 or more probes), such as proximal probes reacted in a sample. In some cases, an oligonucleotide probe may have a reactive sequence on a 3′ end. In some cases, an oligonucleotide probe may have a reactive sequence on a 5′ end. In some cases, an oligonucleotide probe may have a reactive sequence on both the 3′ and 5′ end. The reactive sequences may react with the reactive sequences of other oligonucleotides in the set to form reaction products.

The reactive sequences of a first probe may hybridize to reactive sequence of a second probe, and may be extended along that probe. When the second probe has a hybrization sequence on its 5′ end, the reaction product extended from the first probe may end in a hybridization sequence at its 3′ end, allowing for another round of hybridization and extension along a third probe or another reaction product. Alternatively, reactive sequences may be hybridized to a splint sequence that allows for ligation. Alternatively, reactive sequences may be a transposon.

In certain aspects, oligonucleotides in the set may comprise a variable sequence that, when present in reaction product(s), indicates the relative position of the oligonucleotide with respect to other oligonucleotides in the sample. Target specific oligonucleotide probes, reference oligonucleotide probes, or both target specific and reference oligonucleotide probes may comprise identifier sequences such as variable sequences, which may identify individual occurrences of a probe. In addition, one or more oligonucleotide probes in the set may include at least one identifier sequence such as a target barcode sequence identifying the target an oligonucleotide is specific for, or a characteristic of a reference or target-specific oligonucleotide such as the length of a linker (e.g., PEG linker) of the oligonucleotide. The relative position of some oligonucleotide probes (e.g., probes themselves or probe targets) may be determined through coincidence of identifiers in reaction products.

Reaction mixtures of the subject disclosure may include enzymes and/or oligonucleotides that facilitate the reaction of oligonucleotides in the set to form reaction products. For example, a reaction mixture may comprise reaction reagents, such as a polymerase (and optionally any cofactors and/or dNTPs) that can extend oligonucleotides in the set upon hybridization of their reactive sequences to one another. In certain aspects, reactive sequences may be bidirectional, such that two probes hybridized to one-another at their reactive sequences such that both can extend along one-another in the presence of polymerase. In certain aspects, reactive sequences may be unidirectional (e.g., a terminator sequence on one reactive sequence may result in extension only from the reaction sequence that is not terminated).

In the kits described herein, elements of the kit including one or more sets of oligonucleotide probes (e.g., target specific oligonucleotide probes and/or reference oligonucleotide probes), other reagents for performing the methods described herein, and/or subsets thereof may be provided in admixture or separately, or in any combination.

Methods of the subject disclosure include contacting a sample, such as a biological sample, with a set of oligonucleotide probes. The sample may be incubated with the set of probes under conditions that allow the binding of target-specific oligonucleotide probes to their target. In addition, the sample may be contacted with reference oligonucleotide probes that bind non-specifically throughout the sample. Alternatively, or in addition, reference probes may not bind directly to the sample itself but may be suspended or crosslinked within the sample.

The method may include one or more steps of washing target-specific oligonucleotides and/or reference oligonucleotides bound to (e.g., on or within) the sample.

One or more reaction reagents may be added to the sample after, or in some instances alongside, oligonucleotides in the set. Methods may include reacting proximal oligonucleotides in the set to form reaction products. Reaction products formed in proximity to one another may include the same variable sequence if they reacted with a shared oligonucleotide probe. In certain aspects, the variable sequences of the oligonucleotides in the set are not spatially predetermined (e.g., the location of variable sequences are not known, or are not knowable, independent from the sequences of the reaction products). The reaction may be an extension, ligation and/or an insertion (e.g., by a gene editing enzyme such as a transposase or Crispr). The method may include reacting the oligonucleotides in the set to form reaction products in (e.g. on or within) the sample. In certain aspects reaction products may remain immobilized or bound to the sample as they form and optionally further after they are formed.

In some aspects, one or more reaction products described herein may include 2 or more, 3 or more, 4 or more, 5 or more, 6 or more, 8 or more, 10 or more, 20 or more, 50 or more, 100 or more, 200 or more, 500 or more, or 1000 or more variable sequences from different oligonucleotides within the set. In some aspects, one or more reaction products described herein incorporate sequences (e.g., subsequences) from 2 or more, 3 or more, 4 or more, 5 or more, 6 or more, 8 or more, 10 or more, 20 or more, 50 or more, 100 or more, 200 or more, 500 or more, or 1000 or more variable sequences from different target specific oligonucleotide probes within the set.

Alternatively or in addition, one or more reaction products described herein may include 2 or more, 3 or more, 4 or more, 5 or more, 6 or more, 8 or more, 10 or more, 20 or more, 50 or more, 100 or more, 200 or more, 500 or more, or 1000 or more variable sequences from different oligonucleotides within the set. In some aspects, one or more reaction products described herein incorporate sequences (e.g., subsequences) from 2 or more, 3 or more, 4 or more, 5 or more, 6 or more, 8 or more, 10 or more, 20 or more, 50 or more, 100 or more, 200 or more, 500 or more, or 1000 or more target barcode sequences from different target specific oligonucleotide probes within the set.

Aspects of the subject methods and/or kits may include preparation of the reaction products for sequencing (e.g., library preparation) by any means known by one of skill in the art.

Aspects of the subject methods may include sequencing of the reaction products. The sequences of the reaction products may indicate the relative spatial location of probes (e.g., relative location of probes and/or their targets) in (e.g., on and/or within) the sample. In certain aspects, a reaction product comprising variable sequences from at least 3 or more, 4 or more, 5 or more oligonucleotides in the set indicates the relative spatial proximity of more than two probes on in the sample. Further, the occurrence of the same variable sequence across multiple reaction products may indicate that probes each comprising a different variable sequence present on at least one of the different reaction products were in spatial proximity to one another (e.g., a second order spatial relationship).

Finally, aspects of the invention may include identifying and/or representing the relative spatial relationships of oligonucleotides from the sample, based on their reaction products. In certain aspects, the spatial relationships may be represented as an image (e.g., spatial graph or network). For example, a spatial network may include nodes representing a target bound by an oligonucleotide (or a group of targets) and edges between nodes may indicate variable sequences from oligonucleotides bound to targets of the different node that occur in the same reaction product or group of reaction products. In certain embodiments, first, second and/or third order spatial relationships may be represented.

Aspects of the subject application include a method of identifying spatial relationships within a sample may include identifying the spatial relationship of cells in the sample (e.g., with or without further identifying the spatial relationships of probes or targets in the sample described above). Such a method may include applying spatial barcodes (spatial barcode oligonucleotide sequences, such from spatial barcode probes) to different locations in a tissue sample, wherein instances of a same spatial barcode sequence are applied to a plurality of cells within the same location. Further, a cell at a location may be associated with multiple different spatial barcodes (e.g., spatial barcodes that were applied to an overlapping or partially overlapping location, such as when a spatial barcode is applied directly to a proximal location and then diffuses into the location of the cell). As such, the combination and amounts of unique spatial barcodes associated with (on or in) a cell may together identify that cells location in the sample (e.g., compared to an objective frame of reference such as an optical image or coordinate, and/or relative to other cells in the sample). In certain aspects, the majority of individual cells are each associated with a plurality of different spatial barcode sequences.

Additional method steps described herein may include one or more of dissociation of cells from the sample; isolation of single cells with a cell barcode sequence; lysis of isolated cells; formation of reaction products incorporating spatial barcode sequences; cell barcode sequence, and optionally other target sequences; library prep and sequencing; and identification and representation of single cell localization based on the sequencing data.

In certain aspects, a method of single cell sequencing with spatial resolution may include applying a spatial barcode array to cells in a tissue sample, dissociating cells from the tissue sample, isolating individual dissociated cells in droplets with a bead comprising a cell barcode, lysing isolated cells, incorporating spatial barcodes of an isolated cell with a cell barcode to form individual reaction products that comprise the cell barcode and a spatial barcode, incorporating the cDNA of the isolated cell with the cell barcode to form individual reaction products that comprise the cell barcode and a cDNA sequence, and sequencing the reaction products. The method may further include staining dissociated cells with target specific probes comprising an antibody and target barcode prior to isolating cells, incorporating target barcodes with the cell barcode to form individual reaction products that comprise both a target barcode and a cell barcode sequence. Single cell expression of protein and/or RNA targets may be rendered along with the location of the single cells, based on the sequencing of the reaction products. Aspects include a kit comprising a plurality of the sample barcode probes of any one of the methods described above. For example, sample barcode probes organized on a support and functionalized for attachment to cells in a sample, optionally along with other reagents for binding the sample barcode probes to the sample and/or forming reaction products, may be provided in a kit.

Alternatively or in addition, fragments of tissue (as opposed to single cells) may be dissociated and processed as described in any of the above methods. Further, while a tissue is described in the embodiments above, such embodiments may be modified for any cellular sample in which the location of the cells are of import.

### BRIEF DESCRIPTION OF THE SEVERAL VIEWS OF THE DRAWINGS

FIG. 1 sets forth the relationship between spatial resolution (Y-axis) and multiplexity of target detection (X-axis) in certain exemplary technologies.

FIG. 2A describes an exemplary workflow in which multiple rounds of extension result in reaction products that can be sequenced to identify the spatial relationships between more oligonucleotide probes than are encoded in any single extension product. FIG. 2A shows an initial distribution of oligonucleotide probes in a sample (black horizontal line). Probes comprise hybridization sequences that are complimentary to other probes in proximity, and at least one identifier sequence (e.g., a variable sequence, such as a unique molecular identifier (UMI)). FIG. 2B shows hybridization between proximal oligonucleotide probes. FIG. 2C shows extension from probes hybridized in FIG. 2B. FIGS. 2D and 2E show an additional round of hybridization and extension. Sequencing of all (or some subsets of) the reaction products shown in FIG. 2E allows for identification of the proximity of oligonucleotide probes encoded in the reaction products. Of note, the reaction products AB and FE may extend from other proximal oligonucleotide probes not shown, and additional rounds of extension may occur before sequencing.

FIG. 3A describes an exemplary workflow wherein the 3′ ends of one or more of the oligonucleotide probes are designed to prevent extension but can still act as a template for extending oligonucleotide probes. FIG. 3A shows an initial distribution of oligonucleotide probes, and FIG. 3B shows the result of multiple rounds of hybridization and extension from non-terminated oligonucleotide probes.

FIG. 4A describes an exemplary workflow in a cellular sample. FIG. 4A shows an interior of the cell for purposes of illustration. The reaction could alternatively or additionally take place on the surface of one or more cells. FIG. 4B shows a step of contacting the sample with one or more target specific probes each comprising an affinity reagent (such as an antibody). Unbound probes may be washed away. FIG. 4C shows a step of contacting the sample with reference oligonucleotide probes. The reference oligonucleotide may not comprise an affinity reagent. Steps shown in FIG. 4B and 4C may be performed in any order or simultaneously. The reference oligonucleotide probes may be functionalized to bind covalently to the sample. Individual probes may have different variable sequences. For example, FIG. 4D shows optional addition of a crosslinking agent (e.g., formaldehyde) that could bind the reference oligonucleotide to the sample and/or fix the sample at the same time. FIG. 4E shows extension products after multiple rounds of extension between proximal oligonucleotide probes. Such extension products may be sequenced to identify the spatial relationships between oligonucleotide probes, for example to construct an image (such as a graph). FIG. 4F shows a graph in which edges represent relative spatial proximity of nodes (representing probes) that were in the sample. An edge between two nodes may indicate that variable sequences of probes represented by the two nodes were encoded in the same reaction product (were both present in the same reaction sequence).

FIG. 5 shows certain non-limiting exemplary reagents for use in the subject methods and kits. Reagent 1) is an example of a target specific oligonucleotide probe, specifically an oligonucleotide sequence conjugated to an antibody. The oligonucleotide sequence shows at least one (specifically two) hybridization sequences for hybridizing to other oligonucleotide probe sequences. The sequence also shows a target ID sequence that is specific for the antibody, as well as a spatial identifier (in this case, a unimolecular identifier), which is on the 5′ side of at least one hybridization sequence. Reagent 2) is an example reference oligonucleotides of different length, having different length identifiers. Longer reference oligonucleotides may provide connectivity between clusters of oligonucleotide probes, while short oligonucleotides may provide better spatial resolution within a cluster of oligonucleotide probes. Reference oligonucleotide probes may be functionalized for immobilization, and potentially for even distribution, in the sample (as compared to some target specific oligonucleotide probes). For example, reference oligonucleotides may be functionalized to bind to functional groups (e.g., thiol or amine groups) presented by endogenous molecules, or may be functionalized to be crosslinked within the sample by addition of a crosslinking reagent (e.g., formaldehyde crosslinking of one or more lysines attached to the oligonucleotide). Reagent 3) is an example of a reverse transcription (RT) oligonucleotide probe, which comprises a sequence for extension of endogenous RNA. In this example, another oligonucleotide probe using the RT probe as a template for extension will result in a poly-T 3′ end that can extend from the poly-A tail of endogenous RNA. This allows for sequences (including any identifiers) from both the extending polynucleotide probe and the RT oligonucleotide probe (template) to be on the 5′ end of the reverse transcribed cDNA, allowing for spatial localization of the cDNA.

FIG. 6 shows an exemplary biological sample that can be imaged by the subject methods. In this figure, a particle (such as a bead, nanoparticle or cell) comprising organic molecules (e.g., streptavidin and/or a plurality of cell surface molecules) across its surface may be imaged using oligonucleotide probes. For example, the particle may be a nanoparticle functionalized with streptavidin. The oligonucleotide probes may be functionalized with an affinity reagent (e.g., biotin and/or an anti-streptavidin antibody) or with a functional group for covalent attachment to the organic molecules. After immobilization of oligonucleotide probes to the surface of the particle (as shown in FIG. 6), multiple extensions between proximal probes may be sequenced to identify the spatial relationships of the probes and/or their targets, as described herein (e.g., similar to the process shown in FIG. 4).

FIG. 7 shows distribution of maleimide reactive thiol-groups on the surface of a resting T cell. FIGS. 8A and 8B provides potential elements (FIG. 8A) of probes depicted in later figures and an exemplary probe (FIG. 8B).

FIGS. 9A-C provides an example of a reaction scheme in which probes have two copies of the same complementary sequence, and can extend off probes having the other complementary sequence of the pair.

FIGS. 10A-C provides an example of a reaction scheme in which both complementary sequences of a pair are present on the same probe, such that subsequent extensions alternate which complementary sequence of the pair is at the 3′ end.

FIGS. 11A-C provides an example of a reaction scheme in which a set of probes have a terminator that prevents extension of the terminated probe.

FIGS. 12A to 14C provide examples of reaction schemes in which a probe unbound to sample is reacted with immobilized probes.

FIGS. 15A and 15B provide an example of an ordered reaction scheme in which an extension of a first probe along a second probe ends in a 3′ complementary sequence of a new pair.

FIGS. 16A-D provides a variety of reaction schemes for RNA detection.

FIGS. 17A to 24 provides a variety of exemplary reaction schemes for nested spatial barcodes, in which a set of spatial barcodes are applied across a wider area of the sample (in some cases to predetermined locations).

FIG. 25 provides exemplary reaction schemes in which certain reaction products are enriched prior to sequencing.

FIG. 26 provides a sequencing workflow for identifying locations of cells in a tissue sample.

FIG. 27 provides an example of the FIG. 26 workflow in which a spatial barcode array is applied to a sample.

### DETAILED DESCRIPTION OF THE INVENTION

Described herein are a variety of methods, reagents and kits for determining and/or representing the spatial relationships of oligonucleotide probes (or probe targets) in a biological sample. Such representation may take the form of encoding spatial relationships in oligonucleotide sequences of reaction products, sequencing of spatially encoded reaction products, and analyzing or visualizing spatial relationships based on the spatially encoded sequences. As such, any of the method steps and reagents described in the embodiments set forth herein may individually, or in combination, be used to determine and/or represent such spatial relationships.

### Definitions

Unless otherwise described or inferred in the context of an embodiment discussed below, the description herein uses terms as defined below.

“Oligonucleotide probe”, also referred to as a “probe” or in some cases “oligonucleotide”, comprises a polymer of nucleotides (e.g., natural or unnatural nucleotides). A probe may optionally comprising an additional moiety such as an affinity reagent. Example oligonucleotide probes may bind to a biological sample either covalently (e.g., through a reactive functional group optionally with a bifunctional crosslinker intermediate) or by affinity (e.g., though an affinity reagent intermediary such as an antibody intermediate). In certain aspects, an oligonucleotide is a reaction product of one or more extension, ligation, reverse transcription and/or transposase reactions.

“Terminator” refers to a moiety at the 3′ end of an oligonucleotide probe which prevents extension of the oligonucleotide probe. A terminator may include a non-nucleotide moiety (such as on 3′ Dideoxy-C), or may include a nucleotide (or nucleotide sequence) that is not complementary to a probe that hybridizes to the terminated oligonucleotide probe.

“Sample binding site” may refer to the site of attachment of a probe to the sample, or a portion of the probe that can bind to the sample. For example, a sample binding site may be chemical moiety that binds (covalently or non-covalently) to the sample, or may be an affinity reagent that binds a specific target, as described further herein.

“Target-specific oligonucleotide” or “target specific probe” as used herein is an oligonucleotide that binds to a specific molecule based on hybridization (e.g., to an RNA or DNA target) or affinity (e.g., to a protein). A target specific probe may comprise an affinity reagent that binds based on tertiary structure, such as an antibody (e.g., or fragment thereof), aptamer, lectin, or member of a protein-small molecule binding pair (such as avidin, biotin or a derivative thereof).

“Reference oligonucleotide” as used herein refers to an oligonucleotide that is not specific for a particular target, but is instead distributed throughout the sample such that target specific oligonucleotides can form reaction products with the reference oligonucleotide. Reference oligonucleotides may be immobilized in the sample. For example, reference oligonucleotides may be functionalized to covalently bind a moiety presented by many different molecules or macromolecules in the sample, or to a matrix added to a sample.

“Relative spatial relationships” as used herein means the spatial relationships between probes (e.g., or probe targets), and independent of an objective frame of reference (e.g., a frame of reference external to the probes or probe targets themselves).

“Recursive extension” reactions as used herein refer to the extension of an initial oligonucleotide that hybridizes to and then extends on two or more oligonucleotides in succession. In some of the examples described herein, each of the two or more oligonucleotides has a different variable sequence.

“Variable sequence” as used herein in the context of a group of oligonucleotide probes, is a sequence that is variable within the group of oligonucleotides.

“Barcode sequence” and “identifier” as used herein in the context of a group of oligonucleotides, is a sequence that encodes a unique characteristic of the group of oligonucleotides compared to other groups of oligonucleotides in the assay. Such characteristics may include a target the oligonucleotide is specific for (e.g., as determined by an associated affinity reagent such as an antibody attached to the oligonucleotide), the length of a linker attached to the oligonucleotide, the number of other oligonucleotides in the same group (i.e., set), a location of the group of the nucleotides, and so forth. When a barcode sequence encodes a location of the group of oligonucleotides, the oligonucleotides are introduced to the sample as a packet (e.g., in a droplet, on a bead, from a spot on a solid support). In certain aspects, oligonucleotides sharing the same location barcode are added to the sample without predetermined location, and the location of the oligonucleotides is determined based on sequencing of reaction products formed between multiple groups of oligonucleotides having different location barcodes, or formed between multiple groups of oligonucleotides having different location barcodes and a common target-specific oligonucleotide. In other aspects, the location of the oligonucleotides sharing a location barcode is known beforehand, or determined by an assay other than sequencing of the reaction products.

“Spatial barcode”, “spatial barcode sequence” and “spatial identifier” as used herein refer to an oligonucleotide sequence that is used, or can be used, to identify the spatial location of an oligonucleotide probe in the sample (e.g., a predetermined, known spatial location). A probe with a known spatial barcode may be applied to a known location of the sample, such that the presence of the spatial barcode in a sequenced reaction product indicates the reaction product was formed in that location. Alternatively, spatial barcodes may be applied without prior knowledge of location of individual barcodes, but the relative locations of spatial barcodes (and the targets or cells they bind) may be determined after sequencing reaction products sharing spatial barcodes. In certain aspects, a plurality of oligonucleotide probes in the same location (e.g., region) of the sample may have the same spatial barcode. Such oligonucleotide probes may also include a variable sequence as described herein.

“Nested spatial barcodes” refers to an assay design in which reaction products comprise two, three, four or more types of spatial barcodes, each type of spatial barcode relating to a different spatial resolution. An analogy is identifying a house by zip, city, and street address (three spatial identifiers of different spatial resolution). For example, a first spatial barcode may indicate the location of a reaction product in the sample at the micrometer scale, while second spatial barcode(s) may indicate the location at the nanometer scale.

“Spatially Organized” as used in the context of spatial barcodes refers to the arrangement of oligonucleotide probes applied to a sample or obtained from the sample, where known spatial barcode sequences on the probes relate to predetermined locations on the sample. Unless otherwise specified, probes in the subject kits and methods may comprise variable sequences that are not spatially organized, but that encode the relative spatial relationships of probes (e.g., or their targets). In some cases, kits or methods of the invention may include spatially organized barcodes to identify a general location (or the probes or targets they encode), and spatial barcode(s) that are not spatially organized and indicate the location of the target (or probe, or reaction product).

“Target barcode” refers to a sequence of a probe that identifies the target it specifically binds (such as through an antibody). A probe may have a target barcode and a variable sequence, such that the variable sequence can be used to index (identify) the particular instance of the target bound by the probe.

“Reaction Products” and “reaction sequences” refers to oligonucleotide sequences that have incorporated 2 or more oligonucleotide probe sequences (or subsequences thereof, such as one or more identifiers). In some cases, reaction products may incorporate at least 2, 3, 4, 5, 6, 8, or 10 probe sequences. Reaction products include intermediate reaction products that are still reacting with other probes and/or reaction products.

“Bidirectional extension” refers to reactions in which two hybridized probes extend off each other, while “unidirectional extension” refers to reaction where only one probe extends off the other (e.g., due to a terminator on the other probe). On or both types of extensions may occur in a particular reaction scheme.

“Cleavage” refers to cleavage of a nucleic acid sequence, such as of an oligonucleotide probe, reaction product, or linker of a probe. Cleavage may be chemical (e.g., homolytic or heterolytic), enzymatic (e.g., by a restriction enzyme, or protease for a peptide linker), or by energy (such as by heat, sonication for shearing of DNA or RNA strands, or light for a photocleavable linker).

### Reagents and Kits

Aspects of the subject disclosure include reagents for performing one or more steps of any of the methods described herein. Any combination of one or more reagents disclosed in herein (e.g., in this section, or in the discussion of the subject methods) may be included in a kit.

Reagents may include oligonucleotide probes (e.g., probes containing an oligonucleotide, such as a DNA sequence) that can be applied to a biological sample and reacted with one another to form reaction products comprising reaction sequences that encode the spatial relationship of the probes and/or their targets in (e.g., within and/or on) the biological sample. The probes may have a sequence (i.e., oligonucleotide sub-sequence) that is variable (e.g., random, degenerate) between instances of probes that are otherwise similar. The reaction sequences may comprise a plurality of variable sequences from different probes. One or more of the probes may have a hybridization sequence at one or both ends that hybridizes to and can extend from other probes. Some of the probes may be target-specific probes and may have a target barcode sequence that identifies a target (e.g. an RNA or a protein) that the probe specifically binds to. Other probes may be reference probes that distribute throughout the sample. Reference probes may comprise a reference barcode identifying a property of the reference probe (such as its length of a linker of the probe to the sample).

As described herein, oligonucleotide probes may comprise at least one (e.g., two) hybridization sequences. A hybridization sequence may be at both the S′ and 5′ end of the probe (e.g., allowing for recursive extension). A probe may further comprise one or more identifier sequences. An identifier sequence may be a variable sequence that is specific to a particular instance of the probe. An identifier sequence may be a target specific barcode (target barcode), that identifies a target the probe specifically binds (e.g., through an antibody). The probe may further comprise a target specific moiety, such as an affinity reagent (e.g., an antibody) or a sequence allows for hybridization to a target nucleic acid.

In certain aspects, an oligonucleotide of an oligonucleotide probe is between 10 and 80 nucleotides long, such as between 15 and 50, or 20 and 40 nucleotides long. The oligonucleotide may be more than 10, 15, 20, 30, 40, or 50 nucleotides long. The oligonucleotide may be less than 15, 20, 30, 40, 50, or 80 nucleotides long.

A hybridization sequence of an oligonucleotide probe may be between 4 and 30, between 5 and 20, or between 5 and 15 nucleotides long, or between 15 and 30 nucleotides long. The hybridization sequence may be more than 4, 5, 6, 10, 15, or 20 nucleotides long. The hybridization sequence may be less than 5, 6, 10, 15, 20, or 30 nucleotides long.

An identifier sequence of an oligonucleotide probe may be between 4 and 30, between 5 and 20, or between 6 and 15 nucleotides long. The hybridization sequence may be more than 4, 5, 6, 10, 15, or 20 nucleotides long. The hybridization sequence may be less than 5, 6, 10, 15, or 20 nucleotides long. For example, when the identifier sequence is a variable sequence it may be at least 10 nucleotides long (to reduce redundant variable sequence in different instances of a probe). When the identifier sequence is a target specific barcode, it may be less than 10 nucleotides long, as even 6 nucleotides would allow for hundreds of targets to be barcoded.

In certain aspects, the variable sequence and any target or reference barcode sequence of an oligonucleotide probe may be flanked by hybridization sequences. The hybridization sequences of a probe may be complementary to (may specifically hybridize to) the hybridization sequences of other probes. Hybridization sequences on the 3′ end of a probe may hybridize to, and may extend along, hybridization sequences of another probe. Such an extension may end in another hybridization sequence that can in turn hybridize and extend from another probe or reaction product. Such a reaction may concatemerized multiple probe sequences into a single reaction product. Such a reaction product may encode relative spatial proximity of probes through co-occurrence of variable (and any target or reference barcode) sequences of the probes. As hybridization reactions only occur between probes that are in proximity, and as probes may be uniquely identified by their variable sequences, such reaction products may encode the relative spatial relationships of probes comprising different variable sequences.

Reaction products comprising variable sequences from a plurality of probes may indicate that the plurality of probes were in proximity (e.g., relative spatial proximity) to one another. In other words, a reaction product with 4 different variable sequences may indicate that 4 different probes (each having one of the 4 different variable sequences), were in proximity in the biological sample. If one or more of those different probes also had a target specific barcode sequence, those targets could be identified as being in proximity to one another.

In certain aspects, multiple probes (e.g., or their reaction products) may hybridize to and extend from the same probe. Thus, the variable sequence from that same probe may be incorporated into multiple reaction products, one or more of which may go on to extend from other probes. Therefore, when multiple reaction products comprise the same variable sequence, it may indicate that probes having a variable sequence encoded by one of the multiple reaction products is in some proximity to probes having a variable sequence encoded by another of the multiple reaction products (e.g., at least a second order relative spatial relationship). This can be extrapolated to represent the spatial relationships of all probes in the sample that share reaction products (e.g., directly and/or indirectly).

A target specific oligonucleotide probe may bind to a protein target (e.g., through an antibody bound to the oligonucleotide portion of the probe). A protein specific probe may comprise a target barcode sequence identifying the antibody (or target it is specific for).

A target specific oligonucleotide probe may bind to an oligonucleotide target (e.g., RNA) by hybridization. For example, an RNA specific probe may comprise a poly-T 5′ sequence that hybridizes to the poly-A tail present on an RNA (e.g., on an mRNA) and may be extended along that RNA, thereby encoding its sequence. Alternatively, an RNA specific probe may terminate in a random (e.g., degenerate) 5′ sequence such that it may hybridize and extend from a random RNA sequence. Alternatively, an RNA specific probe may specifically hybridize to a specific sequence, such as a sub-sequence of a target mRNA, and may optionally be extended along that sequence. An RNA-specific probe may comprise a target barcode sequence identifying the RNA target (e.g., specific mRNA) it is specific for, for example in instances where later reaction steps will lose the sequence hybridizing to the RNA.

Some probes may be reference probes, that distribute non-specifically throughout the sample. Reference probes may be functionalized to bind (e.g., covalently bind) to common functional groups presented by biomolecules in the sample. For example, functionalized reference probes (e.g., lysine functionalized reference probes) may be fixed in the sample through addition of a crosslinker (e.g., formaldehyde). Reference probes may have an identifier, such as a variable sequence and/or reference probe barcode.

One or more probes may be immobilized in the sample, such as through binding to a target and/or covalent binding. Probes may be reacted while immobilized in the sample.

In certain embodiments, one or more probes may comprise a particle (e.g., polymer and/or nanoparticle). Such a probe may comprise multiple copies of the same oligonucleotide, such as multiple oligonucleotides comprising the same variable sequence. A first round of extension may create multiple reaction products comprising the same variable sequence. A polymer and/or nanoparticle probe may be synthesized through a process similar to a 1 bead-1 compound library. In such a process, pools of probes (each comprising multiple identical oligonucleotides or a starting functional group) are 1) split into groups, 2) each group is reacted with a different nucleotide composition, 3) groups are mixed into a pool. The process is then repeated to generate particles with multiple oligonucleotide sequences, wherein the oligonucleotide sequences of a particle comprise the same variable sequence, and wherein oligonucleotide sequences of different particles comprise different variable sequences.

In some cases, a kit may include at least 1, 2, 5, 10, 20, 50, 100, 200, 500, or 1000 protein specific probes. In some cases, a kit may include at least 1, 2, 5, 10, 20, 50, 100, 200, 500, or 1000 RNA specific probes. In some cases, as kit may include at least 1, 2, 5, 10, 20, 50, 100, 200, 500, 1000 protein specific probes and at least one RNA specific probe, such as a probe that hybridizes to a poly-A sequence. In some cases, as kit may include at least 1, 2, 5, 10, 20, 50, 100, 200, 500, or 1000 protein specific probes and at least 1, 2, 5, 10, 20, 50, 100, 200, 500, or 1000 RNA specific probes.

Kits for representing spatial relationships of targets in a biological sample may include any reagent, or combination of reagents that are described herein. For example, a kit may include a set (i.e., group) of oligonucleotide probes, wherein a plurality of probes in the group are target specific oligonucleotide probes. The oligonucleotide probes may be reacted to form reaction products comprising reaction sequences that encode the relative spatial relationships between proximal oligonucleotide probes in the sample. One or more of the oligonucleotide probes may be immobilized in the sample when reacted (e.g., may be functionalized to bind to the sample, such as through a covalent bond, affinity reagent, or target hybridization). In certain aspects, the oligonucleotide probes may include reference oligonucleotide probes that are distributed throughout the sample. Reference oligonucleotide probes may be immobilized when reacted.

One or more oligonucleotide probes in the set may include variable sequences. Co occurring variable sequences in a reaction product may encode spatial relationships (e.g., relative spatial relationships) between proximal oligonucleotide probes that included the variable sequences. In certain aspects, different first and second reaction products including a shared variable sequence encodes proximity between oligonucleotide probes that include variable sequences in the first reaction product and oligonucleotide probes that included variable sequences in the second reaction product.

One or more oligonucleotide probes in the set may include a hybridization sequence (e.g., a probe to probe sequence) at their 3′ end. A hybridization sequence of one probe may hybridize to and extend from a hybridization sequence on another oligonucleotide probe in the set (e.g., in a first extension). In certain aspects, the first extension may terminate in a hybridization sequence (e.g., form a reaction product that with the hybridization sequence at the 3′ end) that can hybridize to and extend from another oligonucleotide probes and/or reaction products, allowing for multiple extensions.

In certain aspects, each of the 3′ and 5′ ends of one or more oligonucleotide probes in the set include hybridization sequences to the 3′ and/or 5′ ends of other oligonucleotide probes in the set. For example, the 3′ end of one probe may hybridize to the 3′ end of another probe.

The hybridization sequence at the 3′ and 5′ ends of an oligonucleotide probe may be the reverse complement of one another. Alternatively, hybridization sequences on the 3′ and 5′ ends of an individual probe may be identical, or substantially identical. For example, the hybridization sequences of the 3′ and 5′ ends of an oligonucleotide probe may be identical to each other but may be the reverse complement of the hybridization sequences of other oligonucleotide probes in the set.

Probes may further comprise a variable (i.e., degenerate, random, UMI) sequence. The variable sequence may distinguish an instance (e.g., single molecule) of the probe from otherwise identical probes. The hybridization sequences of a probe may flank the variable sequence.

Probes may further comprise at least one barcode sequence. The barcode sequence may identify (e.g., be associated with) a target that the probe specifically binds to. For example, and barcode may identify a protein target the probe specifically binds to through an antibody intermediate. Such a probe may comprise an antibody covalently bound to an oligonucleotide comprising the barcode sequence. The hybridization sequences of a probe may flank the barcode sequence (e.g., in addition to a variable sequence).

A probe may include a length barcode that identifies a linker of the probe, such as a linker separating an oligonucleotide sequence of the probe from a functional group or affinity reagent for binding to a sample. A length barcode may indicate the length of the linker, such that some probes have different length barcodes that match different linker lengths. Shorter linkers may provide better spatial resolution while longer linkers may allow probes to bridge clusters of probes (e.g., protein islands or complexes) and improve connectivity (e.g., of any graph that could be made based on the reaction products). In certain aspects, “reference probes” that are functionalized to bind or distribute non-specifically throughout the sample may include a linker and length barcode.

In certain aspects, oligonucleotide probes that hybridize to one another may be extended in at least one direction, such as bi-directionally (each probe can be extended along the other after hybridization). Bidirectional extension may improve the rate of reaction and formation of long reaction products, which in turn may improve the hybridization between more distant probes during later stages of the reaction.

In certain aspects, at least some extensions may be restricted to be unidirectional. For example, some oligonucleotide probes in a set may include a 3′ terminator that blocks extension (or ligation) of the probe. Such terminated probes may still act as a template for other probes. Unidirectional extension may allow for a more controlled reaction, reduce amplification biases, and/or allow ordered incorporation of surrounding probe sequences such that order can better inform proximity of probes that are encoded next to (or close to) each other along a reaction product sequence. A terminator may be a bulky side group that prevents extension by a polymerase. While 3′ dideoxy-C is a common example of a terminator, a variety of other terminators are known in the art. The terminator may be a label (e.g., fluorescent label), or may not be a label.

A reaction may start with bidirectional extension but may become unidirectional, such as after one probe extends and terminates in a sequence that cannot rehybridize and extend to other probes. For example, if a portion of probes comprise a 5′ sequencing adaptor sequence (or a portion thereof), or a primer binding sequence for amplification with primers added after the initial reaction, then reaction products will eventually terminate in a sequence preventing further extension (albeit with a range of lengths). For example, some oligonucleotide probes that act as a template for extension (e.g., all probes in a set, or a portion thereof such as probes with a 3′ terminator) may comprise a 5′ sequence that does not reaction product to further extend along other probes. A ratio (such as at or between 0.05 and 0.95, 0.1 and 0.9, 0.1 and 0.4, 0.2 and 0.4) of probes in a set may comprise a 5′ sequence that does not reaction product to further extend along other probes, while the other probes in the set comprise a 5′ sequence that allows further extension along other probes.

One or more of the oligonucleotide probes in the set may be capable of identifying RNA sequences, such as by hybridizing and/or incorporating RNA sequences into their reaction products. For example, one or more of the oligonucleotide probes in the set may include a poly-A sequence at their 5′ end. When incorporated into a reaction product through hybridization and extension from another probe, the reaction product may terminate in a 3′ poly-T sequence that can subsequently hybridize to and extend along a poly-A portion of an RNA target. Reaction products comprising an RNA sequence (or the reverse complement) may be amplified with target-specific primers prior to sequencing. The reaction products produced by oligonucleotide probes in the kit may therefore encode (e.g., identify) the relative spatial relationships of both protein and/or RNA targets.

The kit may allow for the encoding or identification of a plurality of targets in the sample. In certain aspects, reaction products (or their sequences or a spatial representation of their sequences) may encode (e.g., identify) at least 1, 2, 5, 10, 20, 50, 100, 500, 1000 or more distinct protein targets. In certain aspects, reaction products (or their sequences or a spatial representation of their sequences) may encode (e.g., identify) at least 1, 2, 5, 10, 20, 50, 100, 500, 1000, 5000 or more distinct RNA targets (e.g., distinct sequences). In certain aspects, at least 1, 2, 5, 10, 20, 50, 100, 500, 1000, 5000 or more distinct RNA and distinct protein sequences may each be identified. For example, at least 20 protein targets may be identified, such as at least 100 protein targets. In another example, at least 100 RNA targets may be identified. In another example, at least 20 protein targets and 20 RNA targets may be identified, such as at least 100 protein targets and 100 RNA targets. Identification may include identifying a known spatial location of the targets (e.g., relative to one another). In certain aspects, the probes may not need to be handled in a spatially organized manner to create reaction products encoding the spatial relationships between probes. In certain aspects, one or more of the oligonucleotide probes comprise a hairpin structure. The hairpin structures may prevent undesired hybridization and/or may allow for isothermal reactions via strand displacement.

The kit may further comprise one or more additional reagents for at least one of fixing, permeabilizing, forming reaction products, and/or preparing reaction products for sequencing.

In one embodiment, a kit for encoding spatial relationships of targets in a biological sample comprises: a set of oligonucleotide probes, wherein one or more of the probes in the set are target specific oligonucleotide probes, wherein oligonucleotide probes in the kit include a first complimentary sequence at the 3′ end that hybridizes to other oligonucleotide probes in the set; wherein extending from the first hybridization sequence encodes one or more identifiers of the template oligonucleotide probe and terminates in a second hybridization sequence that can hybridize to, and extend along, another oligonucleotide probe in the set; and optionally wherein one or more of the oligonucleotide probes include hairpin structures.

In one embodiment, a kit for encoding spatial relationships of targets in a biological sample may comprise: target specific oligonucleotide probes including an oligonucleotide sequence and an affinity reagent conjugated to the oligonucleotide sequence; wherein oligonucleotide probes in the kit include a first complimentary (e.g., hybridization) sequence at the 3′ end that hybridizes to other oligonucleotide probes in the kit; wherein extending from the first hybridization sequence encodes one or more identifiers of the template oligonucleotide probe and terminates in a second hybridization sequence that can hybridize to, and extend along, another oligonucleotide probe in the kit; and wherein extension products including identifiers from more than one template oligonucleotide can be sequenced to identify the spatial relationships between more oligonucleotide probes than were encoded in a single extension product. The kit may further comprise reference oligonucleotide probes that do not include an antibody. In certain aspects, the identifiers include a variable sequence specific to an instance of a template oligonucleotide probe. The first and second complimentary sequences may be the same or may be the reverse complement of one another. Suitable affinity reagents may be an antibody (or a fragment or derivative thereof), a member of a protein-ligand pair (such as avidin, biotin, or a derivative thereof). The probes may be in admixture.

Certain oligonucleotides may promote strand invasion, for example, to facilitate recursive extensions as described herein under isothermal conditions. In certain aspects, an oligonucleotide including a hybridization sequence for extension may further include a sequence complimentary to a portion of its own sequence, such that it increases the off rate of any sequence extended on the oligonucleotide. In certain aspects, one or more oligonucleotide probes may be hairpins.

Additional reagents may include one or more enzymes for performing any of the methods described herein, such as a polymerase (e.g., DNA polymerase, reverse transcriptase, RNA based RNA polymerase), ligases, restriction enzymes, or transposases. Suitable additional reagents may include buffers, cofactors, and substrates (such dNTPs) for such enzymes. In certain aspects, reagents may include a DNA polymerase and dNTPs. Suitable DNA polymerase may have strand displacement capabilities. In some embodiments, DNA polymerases may have 5′ exonuclease activity. DNA polymerases may be thermostable. When the reactions are isothermal and at a low temperature (e.g., below 60 degrees Celsius), DNA polymerase may not be thermostable.

### Methods

Methods of representing spatial relationships between oligonucleotide probes (e.g., probes themselves or their targets) in a biological sample may include one or more steps described below. Of note, each step may be performed in a number of ways using a variety of reagents. One of skill in the art would understand that certain steps may be optional depending on the application, and additional steps standard in the art may be combined with those described below. The order of the steps described below may be changed provided the method still allows for the imaging of oligonucleotides in a biological sample.

A method of use may include one or more of sample preparation, providing oligonucleotide probes, introducing oligonucleotide probes to a sample, forming reaction products encoding the spatial relationships (e.g., relative spatial proximity) of probes, sequencing reaction products encoding the relative spatial proximity of probes, and analyzing or visually representing the spatial relationships of probes.

Below are example combinations of method steps of certain embodiments. Of note, any of the below methods may include an earlier step of preparing the sample. In certain aspects, the sample may be a biological sample including clusters of proteins, such as a cellular sample that includes at least one cell. Cellular samples include, but are not limited to, cell culture, solid tissue sections, and suspended cells or cell aggregates (such as blood cells, or disaggregated tissue or adherent cell culture). Sample may be prepared by fixation (e.g., by methanol, formaldehyde, etc.) and/or permeabilization (e.g., by methanol or a detergent). The sample may be freshly collected, frozen, or fixed (e.g., Formalin-Fixed Paraffin-Embedded (FFPE) or paraformaldehyde (PFA) fixed). In certain aspects, the sample may comprise a gel matrix, such as a sample enlarged by gel expansion. At least some oligonucleotide probes (e.g., reference probes) may be covalently bound to the matrix.

In certain embodiments, a method of representing the spatial relationships of oligonucleotide probes in a sample may include a) contacting a biological sample with a set of oligonucleotide probes and b) forming reaction products between proximal oligonucleotide probes that include different variable sequences. Steps a) and/or b) may involve use of the reagents or kits described herein, or any element (or combination of elements) of probes described herein. One or more of the oligonucleotide probes may be specific for different targets. One or more of the reaction products may include a plurality of different variable sequences. These variable sequences may encode proximity of oligonucleotide probes when incorporated into different reaction products, such that some separate reaction products include the same variable sequence. For example, a plurality of the variable sequences may be unique molecular identifiers.

The reaction products may encode the proximity of a plurality of oligonucleotide probes. For example, step b) of forming reaction products may include incorporating different variable sequences from at least B, at least 4, at least 5, at least 10, at least 20, at least 50 or at least 100 oligonucleotide probes in one or more reaction products. In certain embodiments, step b) occurs without prior amplification of individual target-specific oligonucleotides probes within the sample. In certain aspects, step b) of forming a reaction product between proximal oligonucleotides does not incorporate any additional variable sequence specific to the formation of that reaction product. In certain aspects, proximal oligonucleotide probes encoded in the same reaction product may have been immobilized in proximity in the sample.

One or more oligonucleotide probes may be reference oligonucleotide probes that are distributed non-specifically in the sample. For example, reference oligonucleotide probes may bind non-specifically to the sample, such as through binding to common functional groups throughout the biological sample. Such reference oligonucleotides may include a linker and optionally a linker barcode (e.g., identifying length of the linker).

One or more of the oligonucleotide probes may be target-specific oligonucleotide probes that specifically bind a target (e.g., RNA or protein). For example, a target-specific probe may include an affinity reagent, such as an antibody (e.g., or fragment thereof, such as the Fc portion of an antibody), that binds to a specific protein target. The oligonucleotide portion of a target specific oligonucleotide probe may include a target identifier sequence (target barcode) that encodes the specific target (e.g., relates to an antibody of the probe). The oligonucleotides in the set may together bind at least 5, 10, 20, 50, 100, 200, 500, or 1000 targets.

One or more of the proximal oligonucleotide probes may be immobilized in the sample during step b) of forming reaction products. For example, one or more of the proximal oligonucleotide probes may be bound to a target through an affinity reagent intermediate (e.g., an antibody, biotin/streptavidin, etc.) during step b) of forming reaction products. Alternatively, or in addition, one or more of the proximal oligonucleotide probes may be covalently bind to the sample during step b) of forming reaction products. One or more of the oligonucleotides probes may be cross-linked to the sample during step b). A method may include fixing one or more of the oligonucleotide probes in the set after step a) of contacting the sample with the set of oligonucleotide probes and before step b) of forming reaction products. For example, certain oligonucleotide probes (e.g., reference probes functionalized with lysine or poly-lysine and/or target specific probes including antibodies) could and cross-linked (e.g., by formaldehyde).

In certain aspects, the reaction products formed in step b) comprise reaction sequences that encode the proximity of protein targets bound by target specific probes in the set during step a). Alternatively, or in addition, reaction products comprise reaction sequences that may encode the proximity of RNA targets (e.g., hybridized during step a) and optionally extended from).

Step b) may include successive extensions (e.g., concatamerization) of a first oligonucleotide probe along multiple additional oligonucleotide probes and/or their extension products. For example, a first extension of a first oligonucleotide probe along a second oligonucleotide probe may form a first extension product. The first extension product may terminate in a 3′ hybridization sequence that then hybridizes to and extends from another oligonucleotide probe (or extension product). In certain aspects, step b) of forming reaction products may be isothermal. Alternatively, step b) of forming reaction products may be through thermal cycling.

The proximal oligonucleotide probes and their reaction products may not be spatially organized based on their variable sequences. In certain aspects, the probes may not be spatially arranged in any deterministic fashion prior to formation of the reaction products. As such, the spatial relationships between probes represented by the co-occurrence of variable sequences in reaction products may be relative to one another, and not be based on an objective frame of reference (such as a coordinate system). In certain aspects, the reaction products comprise reaction sequences that may together encode the relative proximity between of at least 10, at least 20, at least 50, at least 100, at least 200, at least 500, or at least 1000 probes (e.g., oligonucleotide probes and/or their targets).

The variable sequences of a single oligonucleotide probe may be present on different reaction products, and a plurality of the different reaction products may each include a distinct combination of variable sequences. In certain aspects, the combination of variable sequences on a plurality of the different reaction products includes at least 4 different variable sequences.

DNA is an extremely stable medium. As such, reaction products having the properties described herein, may be stored indefinitely before or between sequencing of the reaction products.

A method may include, or may further include, step c) of sequencing reaction products encoding the spatial relationships (e.g., relative spatial proximity) of probes in a sample. Suitable forms of library preparation for sequencing are known in the art. Library prep may be performed in the sample, or after reaction products are removed (extracted from) the sample. Sequencing may cover all, or only part, of the full reaction product sequence. Sequences of reaction products that include multiple different variable sequences may indicate relative spatial proximity (in the sample) of the probes comprising the variable sequence.

A method may include, or may further include (e.g., in addition to step c of sequencing, or in addition to all of steps a, b and c), a step d) of representing the relative proximity of oligonucleotide probes. Representing may include visually representing the relative proximity of oligonucleotide probes and/or their targets based on the sequences of their reaction products. In certain aspects, the relative proximity of probes may be visually represented as a graph. Nodes in the graph may represent a variable sequence (e.g., a probe including the variable sequence and/or a specific target of the probe), and edges between nodes represent the co-occurrence of variable sequences in at least one reaction product. In certain aspects, the spatial relationships represented (e.g., encoded) by the reaction products and/or by analysis of the sequences of the reaction products, represent spatial relationships between probes (e.g., probes themselves and/or their targets) in the sample (e.g., as positioned on or in the sample during step a) and/or b)). In certain aspects, the spatial relationships may be on the nanometer scale. For example, one or more of the represented spatial relationships may be 100 nm or less, such as between 10 and 100 nm. The represented spatial relationships may a 2D distribution of oligonucleotide probes and/or their targets, or a 3D distribution of probes and/or their targets.

Edges may be arranged or weighted to represent the strength (e.g., proximity) of spatial relationships. For example, multiple first order associations (co-occurrence of probe sequences in a plurality of different reaction products) may indicate a close spatial proximity over just a single first order association. A second or third order association with a lack of any first order association may indicate a more distant spatial proximity. In certain aspects, edges of the graph may be pruned, e.g., such that a first order association may be ignored when not reinforced by additional associations.

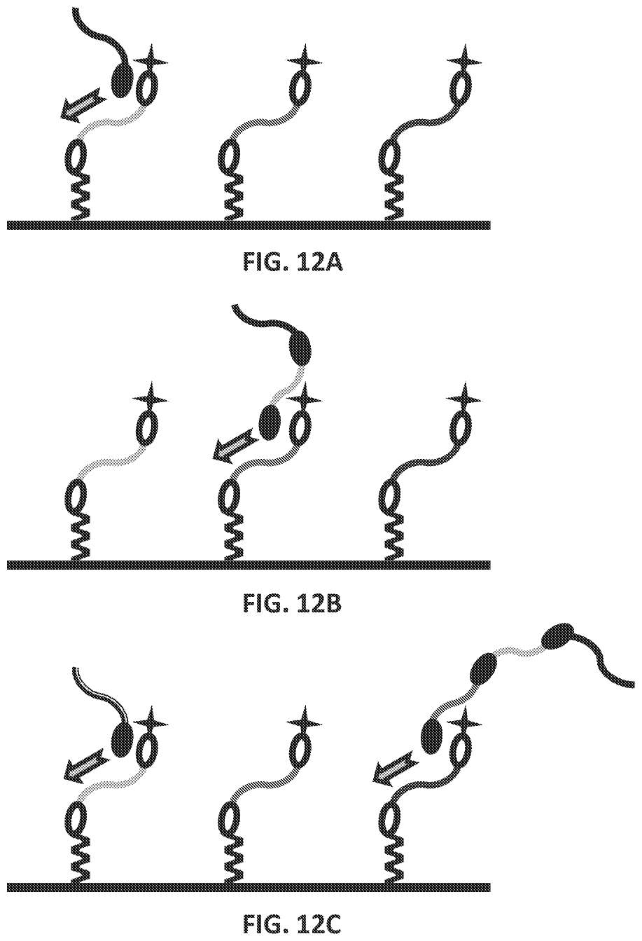

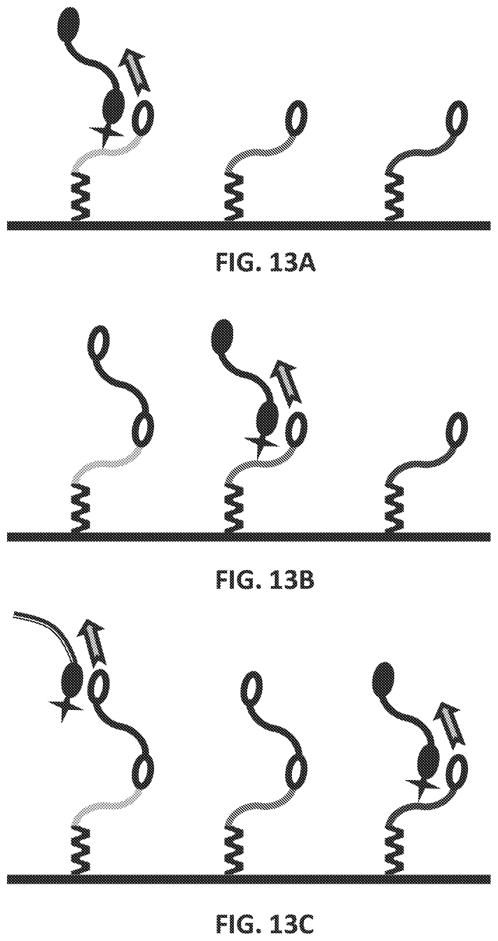

In certain aspects, the order of probe sequences in a reaction product may indicate the proximity those probes were in while bound to the sample, e.g., such that probe sequences that are closer to each other on a given reaction product indicate the probes were likely in greater proximity. For example, an unbound probe (unbound to sample) may extend along template probes bound to the sample in a series of unidirectional extensions such that the diffusion of the unbound probe between two extensions is less than the overall area of diffusion of the probe across all extensions (e.g., as shown in FIGS. 12 to 14).

A method of representing the spatial relationships of oligonucleotides in a biological sample, may include sequencing the reaction products encoding the relative spatial relationships of oligonucleotide probes, and constructing a visual representation of the spatial relationships of at least 100 probes, based on the sequence of the reaction products (e.g., and not on an external frame of reference). In certain aspects, the visual representation is a graph (e.g., nodes connected by edges).

When two oligonucleotides include variable sequences present in the same sequenced reaction product, oligonucleotide nodes representing the two oligonucleotide sequences may be connected by an edge. Spatial relationships are represented as edges between nodes, such that each of the at least 100 oligonucleotide nodes are connected all other nodes directly and/or indirectly (through connection to intermediate nodes). For example, at least two nodes may be connected indirectly through one or more additional nodes. As such, the graph may represent first, second and third order spatial relationships. Clusters of nodes (nodes that share the strong proximity) may be represented as a single node in a larger field of view.

One or more of the oligonucleotides probes may have been hybridized to target nucleotide sequence endogenous to the biological sample prior to the step of sequencing. The hybridized oligonucleotide probe may further have been extended from the target nucleotide sequence. In certain aspects, the target nucleotide sequence may be an mRNA sequence. Alternatively, or in addition, a plurality of the oligonucleotides may have been bound to a protein target through an antibody intermediate.

Aspects of the invention include a method of sequencing reaction products formed between proximal oligonucleotides in a biological sample, the method including a) contacting the biological sample with a plurality of reference oligonucleotide probes, wherein each reference oligonucleotide probe includes a variable sequence; b) contacting the biological sample with a plurality of target specific oligonucleotide probes each bound to an affinity reagent, wherein each target specific oligonucleotide probe includes an affinity reagent ID sequence; c) forming reaction products between proximal reference oligonucleotide probes and target specific oligonucleotide probes, such that one or more of the reaction products include three or more variable sequences; and optionally d) sequencing the reaction products. In certain aspects, the target specific probe further includes a variable sequence. One or more of the probes may be immobilized in the sample during step c) of forming reaction products.

A method of forming reaction products by recursive extension may include a) contacting a biological sample with a plurality of oligonucleotide probes, wherein each oligonucleotide probe includes a variable sequence and at least one hybridization sequence; b) contacting the biological sample with a polymerase; c) hybridizing the hybridization sequences to their complement on proximal oligonucleotide probes; d) extending proximal oligonucleotide probes from the hybridization sequences; and e) repeating steps c) and d) at least once, such that each of a plurality of oligonucleotide probes is extended to form a recursive extension product that incorporates the variable sequences of three or more proximal oligonucleotide probes.

One or more of the oligonucleotide probes may be immobilized on the biological sample prior to step c) of hybridizing. In certain aspects, step d) of extending may form a 3′ hybridization sequence, or form a 3′ complement to the hybridization sequence, that is extended from in step e). Some of the oligonucleotide probes may further include a second instance of the hybridization sequence or its complement, for example, at the 5′ end of the probe. Some of the oligonucleotide probes further include an adaptor sequence, such that one or more of the recursive extension products are suitable for sequencing. Alternatively, some of the oligonucleotide probes may include primer binding sites for additional amplification of recursive extension products with primers added in a later step. Such additional amplification may incorporate sequencing adaptors. Methods may include hybridizing a sequencing primer to at least a portion of an adaptor of a reaction product and sequencing the reaction product in a sequencing by synthesis reaction.

A method may further include heating the biological sample to dehybridize extended proximal oligonucleotides prior to step e). In certain aspects, hybridization in step e) is facilitated through strand invasion (e.g., such that heating is not required to dehybridize prior to the next extension. As such, the method may include maintaining a lower temperature (e.g., 25 degrees Celsius or less, 37 degrees Celsisus or less, 42 degrees Celsius or less, 45 degrees Celsius or less), such that step e) is isothermal.

In certain aspects, a method of encoding the proximity of several targets on individual reaction products may include a) hybridizing the 3′ end of a first oligonucleotide to a second oligonucleotide; b) extending the first oligonucleotide probe along the second oligonucleotide to form a first extension product; c) hybridizing the 3′ end of the first extension product to a third oligonucleotide; d) extending the first extension product along the third oligonucleotide to form a second extension sequence, wherein a plurality of the oligonucleotides include a variable sequence; and e) hybridizing the 3′ end of the second extension sequence to a fourth oligonucleotide sequence. The method may further include step f) of extending the second extension product along the fourth oligonucleotide sequence to form a third extension sequence. In some cases, the third oligonucleotide sequence may be a probe, or a reaction product formed from two or more other probes. The method may further include sequencing the reaction products. The method may further include identifying spatial proximity between different oligonucleotides based on the co-occurrence of variable sequences within sequenced reaction products.

As used herein, “proximity” may be used to describe the range across which a probe react with other probes to form reaction products, or may be used to describe the distance estimated to be between two probes encoded in the same reaction product(s). In general, proximity is the resolution of a particular assay. As such, proximity may be less than 1 mm, less than 500 urn, less than 200 urn, less than 100 urn, less than 50 urn, less than 20 urn, less than 10 urn, less than 5 urn, less than 2 urn, less than 1 urn, less than 500 nm, less than 200 nm, less than 100 nm, less than 50 nm, less than 20 nm, or less than 10 nm depending on the assay design.

### Sample Preparation

A sample for use in the subject methods may be any biological material. In some cases, a biological sample may comprise cells. A sample may be obtained from cell culture or from tissue of a host (e.g., an animal model or a human patient). The sample may be solid (e.g., an adherent cell culture or solid tissue) or disaggregated (e.g., cells or tissue fragments in suspension). Other examples of biological samples include beads or another solid surface comprising biological molecules (organic molecules with biological activity).

A sample may be prepared (or may have been prepared) by fixation. Alternatively, or in addition, the sample may be fixed during or after the step of contacting described above. In some cases, a sample may also be wax embedded, such as a formalin-fixed paraffin embedded (FFPE) sample.

Alternatively, or in addition to fixation, a sample may be permeabilized, such as by a detergent (e.g., Triton or saponin) or an alcohol (e.g., methanol). Permeabilization may allow probes to better penetrate the sample (e.g., penetrate the cell membrane to bind to intracellular targets). Permeabilization may be performed before and/or during the contacting step.

A sample may be expanded by gel expansion. In certain cases, gel expansion may increase spatial resolution. A gel matrix may provide anchor points for reference probes.

Aspects of the invention include a sample contacted with oligonucleotide probes as described herein. Aspects of the invention also include a sample comprising reaction products formed from oligonucleotide probes.

### II. Introducing Oligonucleotides Probes

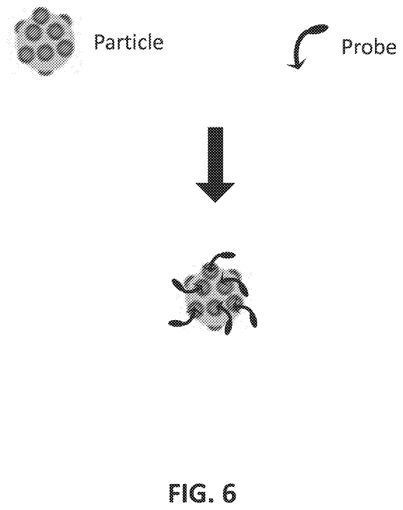

A sample (e.g., biological sample) may be contacted with oligonucleotide probes (e.g., a set of oligonucleotide probes). Certain oligonucleotide probes may bind to specific targets (e.g., specific proteins) in the sample, while other oligonucleotide probes may distribute throughout the sample non-specifically (such as through binding to a common chemical moiety). For example, a particle (e.g., cell or bead) may be contacted with oligonucleotide probes, such as is shown in FIG. 6.

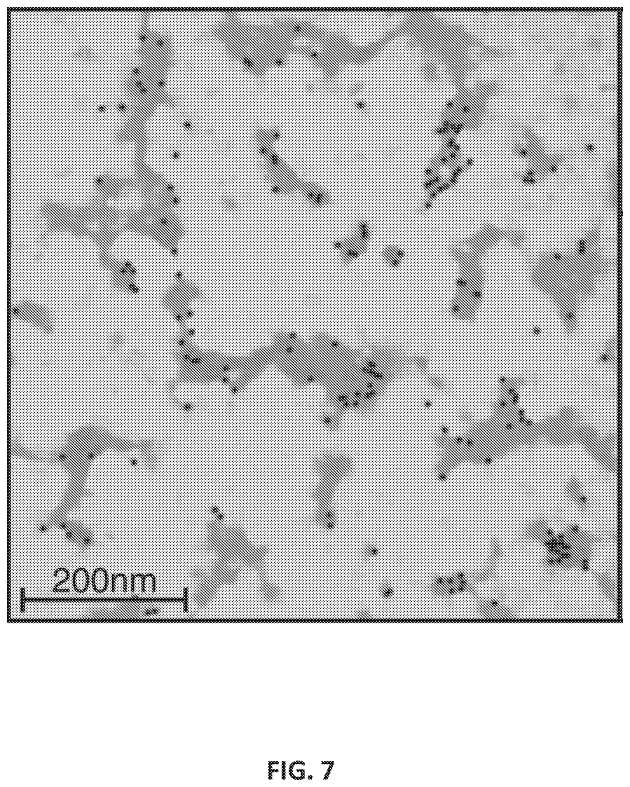

FIG. 7 shows distribution of maleimide reactive thiol-groups on the surface of a resting T cell. Image was published by Lillemeier et al. in a scientific publication entitled “Plasma membrane-associated proteins are clustered” (Proceedings of the National Academy of Sciences 103.50 (2006): 18992-18997). Briefly, biotinylated thiol groups on the cell surface via a maleimide intermediate, followed by binding of biotin by streptavidin Au nanoparticles. A portion of the cell surface was imaged by electron microscopy. The distance between nanoparticle and its closest neighbor ranges from a few nanometers to tens of nanometers. As such, reference and/or target oligonucleotide probes immobilized with a similar distribution across the surface of the cell could extend from proximal oligonucleotide probes (e.g., on the same cluster or protein island), and a longer oligonucleotide probe (e.g., with a linker or after multiple extensions) could span tens of nanometers (e.g., between clusters or protein islands).

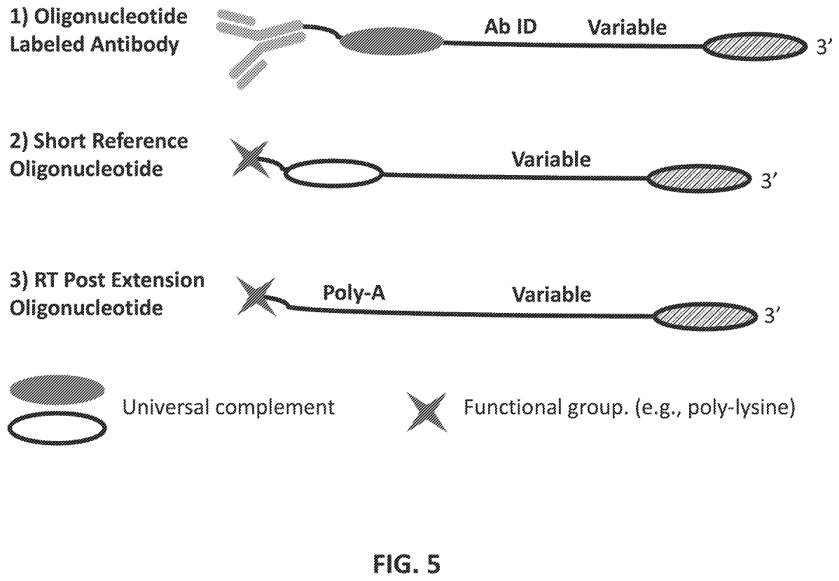

FIG. 5 shows exemplary oligonucleotides of the subject methods and kits. As shown in FIG. 8, oligonucleotide probes may include any combination of a number of elements and/or properties. Exemplary elements shown in FIG. 8A include a sample binding site, variable sequences, complementary sequences, terminator, and additional sequences such as spatial barcode, sequencing adaptor, binding sites for additional primers, target barcode sequences, and/or additional instances of the aforementioned elements. FIG. 8A also shows properties of probe reactions, such as direction of extension and cleavage of probes. FIG. 8B shows an exemplary probe with a site where it binds to a sample, a variable sequence (with optional additional sequences), complementary sequences, and a terminator. Of note, the two complementary sequences of the probe shown in FIG. 8B be identical, or may together be a pair of complementary sequences (may be complementary (e.g., the reverse complement) to each other). These elements and properties are described in detail elsewhere.

In certain aspects, a sample binding site of a probe may be a chemical moiety such as a functional group. The functional group may be a reactive group that binds covalently to moieties presented by the sample. For example, maleimide may be a bound to thiol groups presented by proteins in a sample. In certain aspects, some probes may be reference probes that distribute throughout the sample without binding to any particular protein or oligonucleotide target. A sample binding site may be a chemical moiety that binds non-covalently to sample, such as through lipophilic interaction with cellular membrane. A sample binding site may be a functional group that can be bound covalently to the sample through an intermediate such as a fixative (e.g., formaldehyde fixation of a lysine functional group of a probe to other amines in the sample) or a matrix.

In certain aspects, a sample binding site of a probe may be an affinity reagent described herein, such as an antibody (or fragment or derivative thereof). An affinity reagent specifically binds a unique target, such as a particular protein, such as through interaction with its tertiary structure.

In certain aspects, a sample binding site of a probe may be a sequence that specifically hybridizes to nucleic acid (e.g., an RNA or DNA sequence) of the sample, such as a poly-A tail or a target specific sequence.

In certain aspects, a sample binding site may include a linker (e.g., PEG linker) to space probes away from the site of binding to the sample (and allow for reaction with more distant probes). As described herein, oligonucleotide probes may comprise at least one (e.g., two) hybridization sequences. A hybridization sequence may be at both the 3′ and 5′ end of the probe (e.g., allowing for recursive extension). A probe may further comprise one or more identifier sequences. An identifier sequence may be a variable sequence that is specific to a particular instance of the probe. An identifier sequence may be a target specific barcode (target barcode), that identifies a target the probe specifically binds (e.g., through an antibody). The probe may further comprise a target specific moiety, such as an affinity reagent (e.g., an antibody) or a sequence allows for hybridization to a target nucleic acid. Any probe described herein, such as in any of the subject kits, may be introduced to a biological sample.

In certain aspects, a sample may be adhered to a solid support (e.g., a slide). Alternatively, a sample may be suspended in solution.

Oligonucleotide probes (e.g., a set of oligonucleotide probes) may be introduced to (put in contact with) a sample alongside any other reagents to allow probes to bind their targets. Such reagents may include a buffer (e.g., providing physiological conditions for antibody binding) and/or permeabilization reagents (e.g., to allow probes that specifically bind intracellular targets to enter the cell). Alternatively, in the absence of permeabilization, probes may not penetrate the cell membrane, and reaction products may therefore only indicate spatial proximity of extracellular and/or cell surface targets.

The step of introducing oligonucleotide probes may further include washing unbound oligonucleotide probes from the sample.

The step of introducing oligonucleotide probes may further include contacting the sample with a crosslinking (e.g., fixation) reagent that immobilizes one or more oligonucleotide probes in the sample. Crosslinking may be performed alongside or after introducing the oligonucleotide probes to the sample (e.g., before a wash step and/or after a wash step).

### Spatially Prearranged Probes

Reaction products may also incorporate a predetermined spatial barcode (a barcode sequence that identifies a known location in the sample). As such, oligonucleotide probes (e.g., reference probes described herein) may be applied to the sample in a spatially prearranged matter (e.g., in the form of an array), such that their position in the sample corresponds with a known spatial barcode. These probes may also include a spatial barcode sequence that does not relate to their spatial prearrangement. Alternatively, other probes may include a spatial barcode sequence that does not relate to their spatial prearrangement. Reaction products may include at least on spatial barcodes in addition to two or more variable sequences from different probes, such that the spatial barcode can identify a general location (e.g., region) of the different probes and the variable sequences can be used to identify spatial proximity of probes (or their targets) within that location.

### III. Reaction

Oligonucleotide probes described herein may be reacted in (e.g., on) a biological sample to form reaction products that incorporate the variable sequences of proximal oligonucleotides.

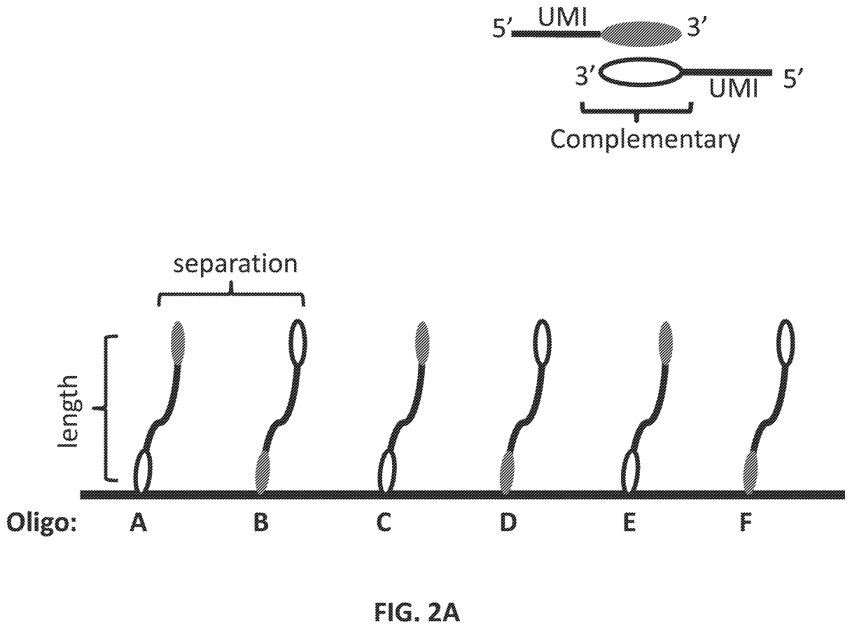

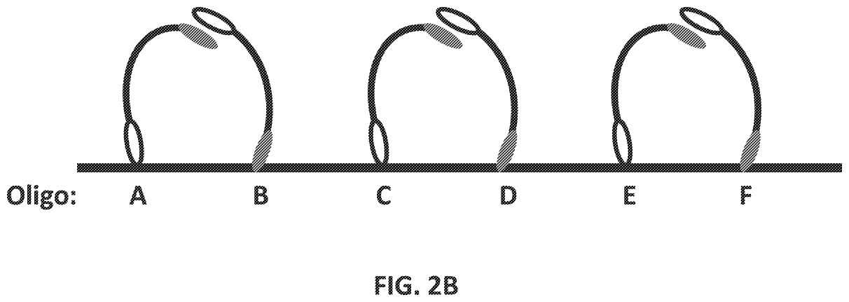

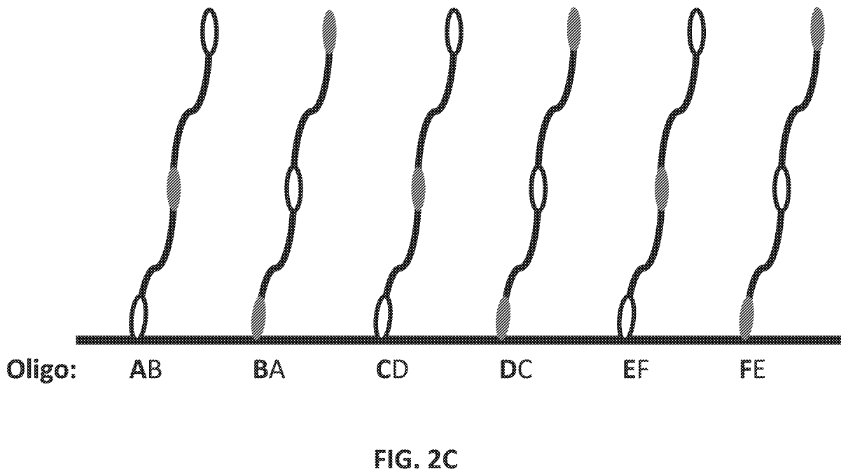

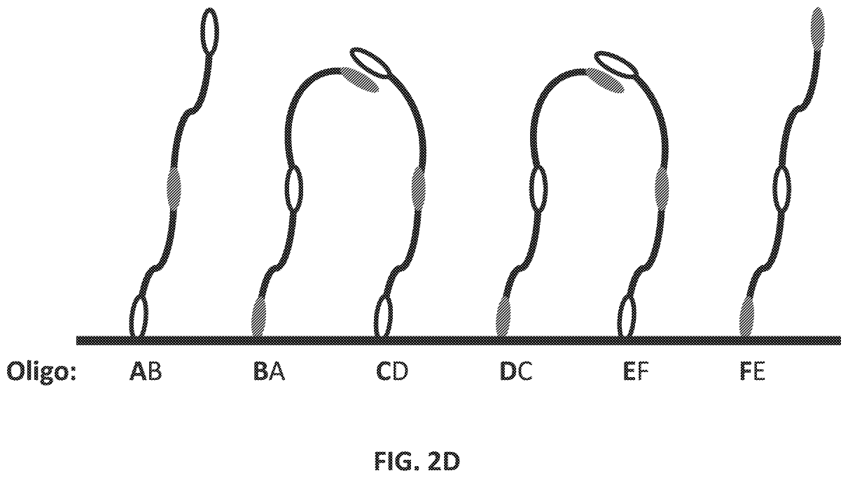

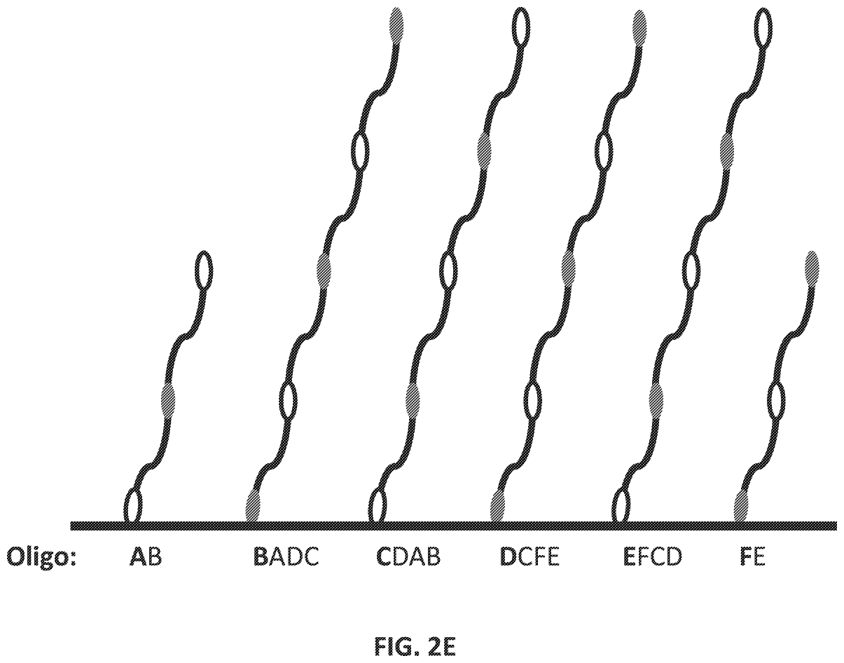

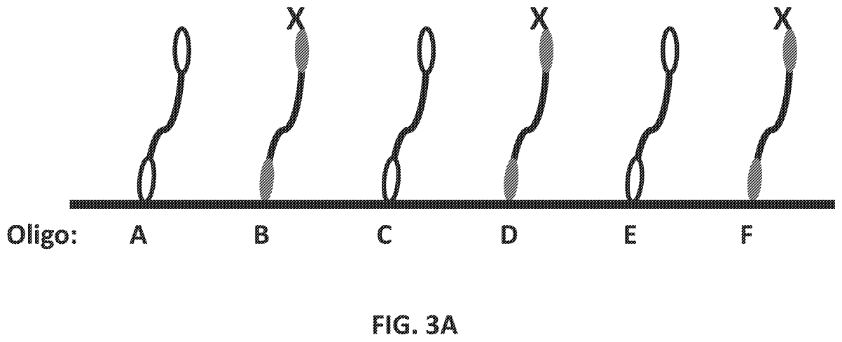

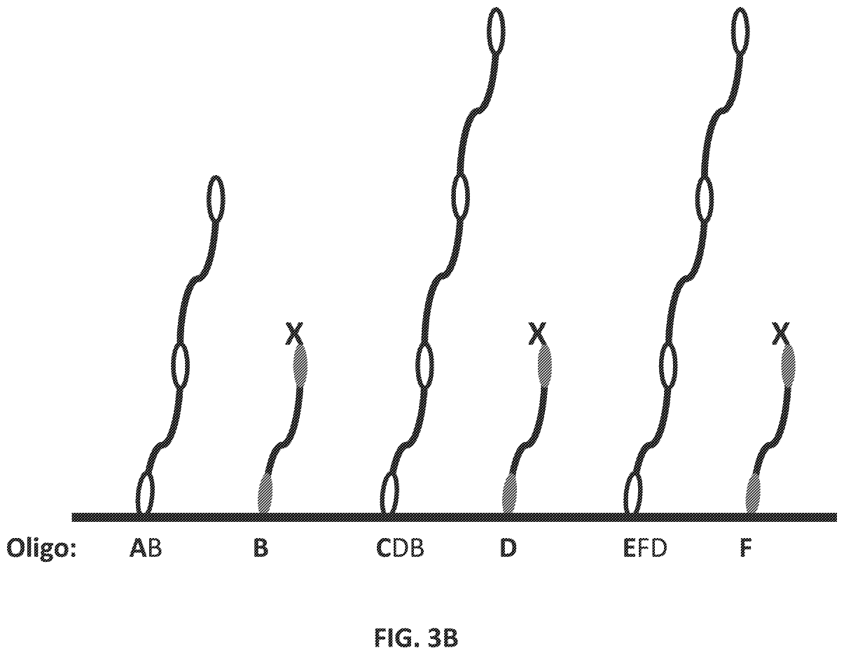

FIGS. 2 and 3 show exemplary reaction schemes described earlier. FIG. 8A, and subsequent figures of exemplary reaction schemes, use arrows to indicate direction of extension (e.g., unidirectional or bidirectional extension). Cleavage is also depicted in some reaction schemes.

In certain embodiments, reaction products have at least 3 or more, 5 or more, 8 or more, 10 or more, 15 or more, 20 or more, 3 to 20, 3 to 15, 3 to 10, 3 to 8, 5 to 20, 5 to 15, or 5 to 10, 8 to 20, or 8 to 15 different variable sequences. For example, the majority of reaction products may have at least 3 or more, 5 or more, 8 or more, 10 or more, 15 or more, 20 or more, 3 to 20, 3 to 15, 3 to 10, 3 to 8, 5 to 20, 5 to 15, or 5 to 10, 8 to 20, or 8 to 15 different variable sequences. The co-occurrence of variable sequences in reaction products may indicate proximity of oligonucleotide probes (e.g., or their targets) in the sample that included the co occurring variable sequences.

As described herein, a first oligonucleotide probe in a set may act as a template for a second oligonucleotide probe in the set when matching (reverse complement) sequences of the two probes hybridize, and the second probe extends along the first probe. Over multiple extension steps, a probe that extended from a first template probe may move to hybridize and extend from a second template probe. In certain aspects, two probes may both reciprocally act as the template for one another. A probe (e.g., or its reaction product) may move from one template (e.g., another probe or reaction product) to another in between hybridization (and extension) steps. A first reaction product (e.g., extended from one or more oligonucleotide probes) may have multiple hybridization sites (e.g., 3, 4, 5, 6 or more) that allow a plurality of other probes (or their reaction products) to hybridize and extend from. The location of a first match (first hybridization of one probe to a second probe) may determine the area in which it finds another probe to hybridize to at a later step. Further, elongation through multiple (i.e., recursive) extensions may further determine which probes are in reach of the extended probe (e.g., reaction product). When an extension terminates in the reverse complement of the sequence that hybridized to initiate it, the next extension may alternate (hybridize to the reverse complement of the sequence hybridized to in the earlier extension). In certain embodiments, a probe may actively extend across multiple template probes either in subsequent steps (e.g., subsequent thermal cycles) or in the same step (e.g., isothermally). Some hybridizations may result in no extension, for example when the 3′ end of a probe (e.g., or its reaction product) is hybridized to the 5′ end of another probe (e.g., or its reaction product).

In certain aspects, a reaction step may further include preparation of reaction products for sequencing. Such preparation for sequencing may include isolating the reaction products from the sample, for example by washing the reaction products out of the sample or disrupting the sample (e.g., by lysis), and optionally further by purification of reaction products (e.g., on DNA binding beads and/or alcohol purification). Preparation may also include preamplification of reaction products. Preparation for a specific type of sequencing, such as lllumina sequencing by bridge amplification, may also include incorporation of adapter sequences and/or sample identifiers as known to one of skill in the art.

### Exemplary Reaction Schemes

Additional reaction schemes described below illustrate reaction schemes that may vary by complementary sequences, direction(s) of extension, and which probes are immobilized. Variations in these reaction schemes provide reaction products that may vary in the spatial resolution of their encoded spatial relationships. Of note, FIG. 8A provides potential elements of probes and reactions depicted in later figures. Most of the reaction schemes described herein (or portions thereof) may be combined to provide a combination of the benefits provided by the individual reaction schemes.

For example, some reaction schemes allow extension of a reaction product from itself. Resultant reaction products may stochastically enrich for variable sequences or combinations of variable sequences, which could then be used as a spatial zip code as described herein.

Some reaction schemes may include unidirectional extension. Resultant reaction products may encode proximity in the order of variable sequences.

Some reaction schemes may include some probes that are immobilized, which may increase spatial resolution. Including at least some probes that are not immobilized may increase the distance across which spatial relationships can be represented.

Reactions may be run isothermally or through thermal cycling. For example, a plurality of complementary sequences on proximal oligonucleotide probes and/or their reaction products may promote strand invasion to allow for multiple extensions under isothermal conditions. Optionally, an excess of a complementary sequence may be provided in solution to disrupt prolonged hybridization of probes and/or reaction products, thereby improving rate of multiple extensions under isothermal conditions and/or lower temperatures (such as less than 25, 32, 37, 42, or 45 degrees Celsius).

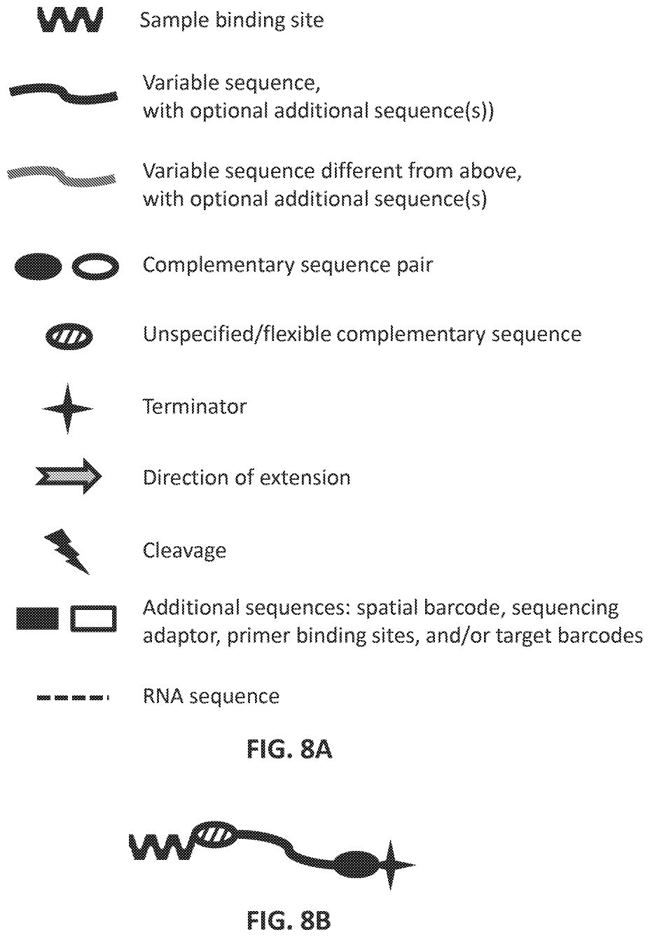

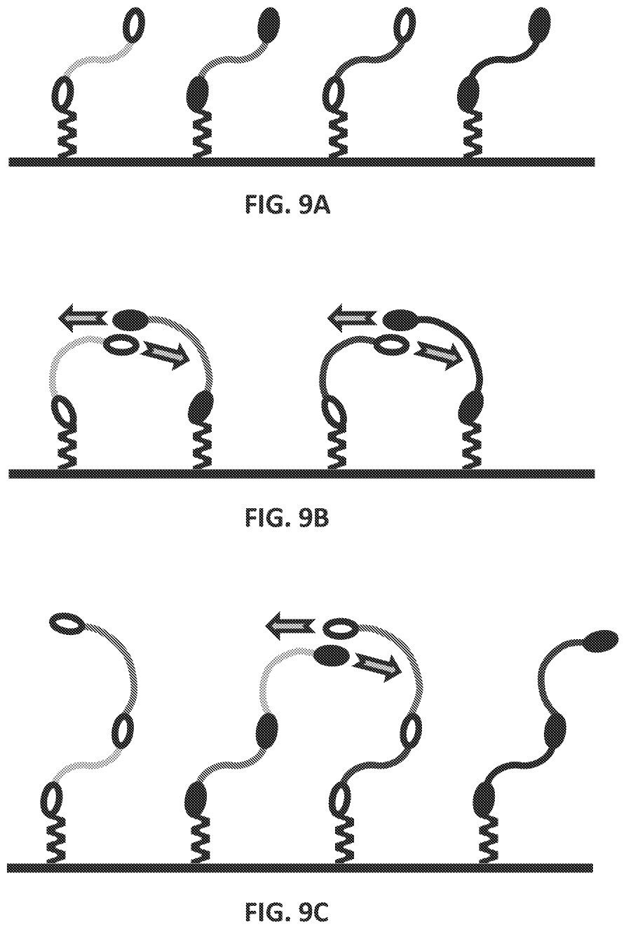

FIG. 9 provides an example of a reaction scheme in which probes have two copies of the same complementary sequence, and can extend off probes having the other complementary sequence of the pair. The horizontal black line represents the sample probes are immobilized to (as it does in subsequent figures). FIG. 9A shows immobilization (binding) of probes to the sample. FIG. 9B shows a first hybridization and extension step. FIG. 9C shows the reaction products resulting from the step shown in FIG. 9B, and shows the hybridization of the reaction products to one another and additional extension steps. Of note, reaction products may hybridize to and extend from probes that have not yet been extended. As described further herein, sequencing the reaction products may allow the identification of proximity of probes (e.g., probe targets) in the sample based on coincidence of variable sequences the same reaction product. A variable sequence (from the same probe) that occurs in multiple reaction products can be used to determine some proximity of probes with variable sequences that occur in different reaction products of the multiple reaction products. As such, proximity (and distribution) of probes can be build out in a relativistic manner (e.g., represented by a graph) as described further herein.

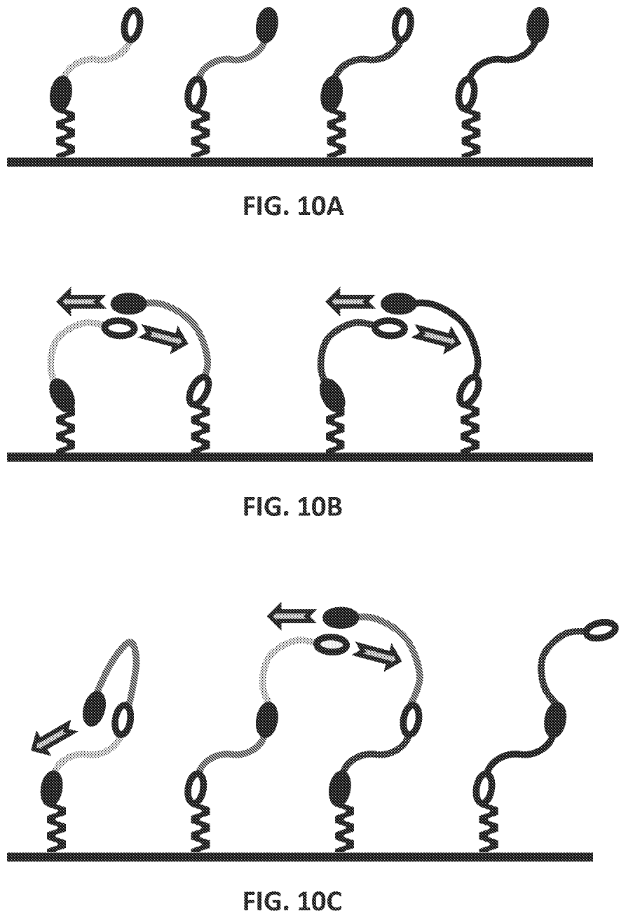

FIG. 10 provides an example of a reaction scheme in which both complementary sequences of a pair are present on the same probe, such that subsequent extensions alternate which complementary sequence of the pair is at the 3′ end. Of note, this reaction scheme may be inhibited by hairpin formation. However, formation of a hairpin such as is shown in FIG. IOC (left reaction product) may allow for additional extension of the probe. Reaction products that have multiple instances of the same complementary sequence, and at least one instance of the other complementary sequence of the pair, may exhibit strand invasion that displaces hybridized reaction products (allowing them to hybridize to and extend from another probe or reaction product). One benefit to this scheme may be that reaction products are able to react with all other reaction products.

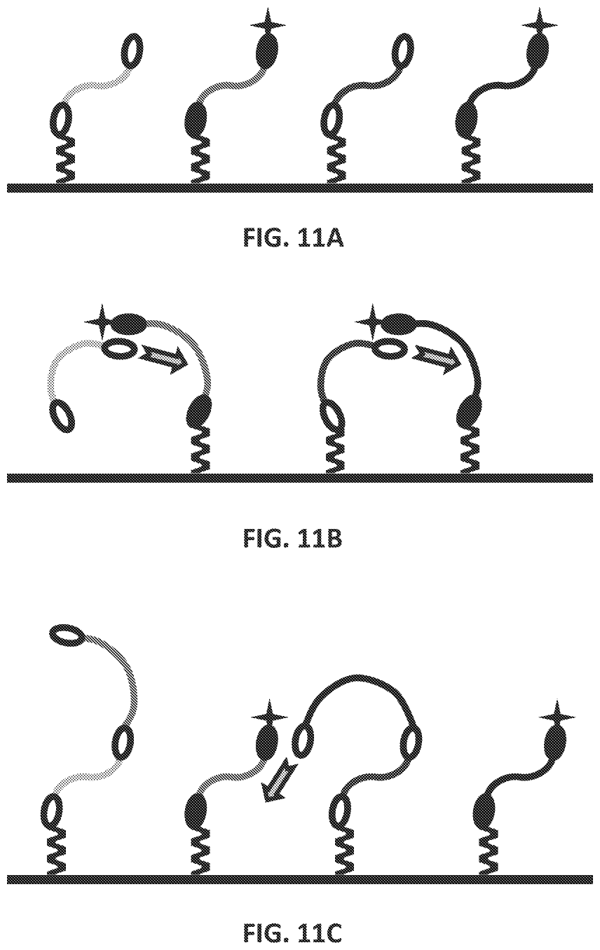

FIG. 11 provides an example of a reaction scheme in which a set of probes have a terminator that prevents extension of the terminated probe. In this example, the set of probes with the terminator group have the same complementary sequence. This may prevent bidirectional extension (enforce unidirectional extension), allowing for a more controlled reaction. For example, there may be less amplification bias and/or better correlation between proximity of probes (e.g., or their targets) and coincidence of their sequences on the same reaction product. Another benefit may be the ordered addition of probe sequences, in which proximal probe sequences on a reaction products may better indicate proximity than in more chaotic bidirectional extension reaction schemes.

FIGS. 12 to 14 provide examples of reaction schemes in which a probe unbound to sample is reacted with immobilized probes. In FIG. 12, the unbound probe extends along bound probes that have a terminator. In FIG. IB, the unbound probe is terminated such that extension only occurs from the bound probe. In FIG. 14, both the unbound probe and bound probes can extend bi-directionally.

An unbound probe (e.g., probe without a sample binding site) may diffuse between extensions. In a sample (e.g., tissue, cell, and/or gel matrix) and in the presence of complementary sequences presented by sample bound probes, this diffusion may be limited. For example, unbound probes and/or their reaction products may diffuse (on average) less than 10 urn, less than 5 urn, less than 1 urn, less than 500 nm, less than 200 nm, or less than 100 nm before hybridizing to (and extending from) the next probe or reaction product. In certain aspects, a long unbound reaction product (e.g., that has incorporated more than 3, more than 5, or more than 10 different probe sequences) may “walk” across the sample (e.g., such that at least part of the reaction product is hybridized to bound probe or bound reaction products the majority of the time that the reaction product moves across the sample). Such a long unbound reaction product may form from a reaction scheme such as that shown in FIG. 12 or FIG. 14.

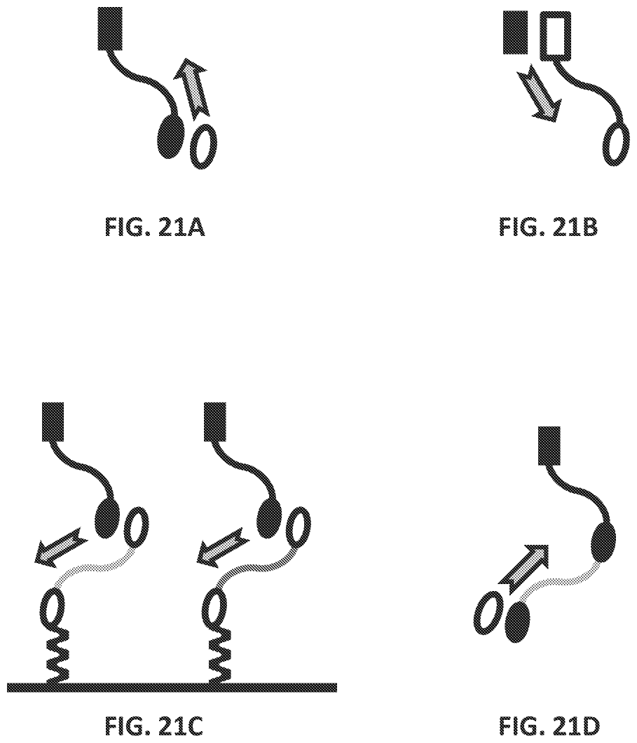

While the unbound probes shown in FIGS. 12 to 14 are shown to have an element representing variable sequence (and optionally additional sequences), it is not a necessary element, as variable sequences may be provide only by bound probes. Alternatively, some or all of the bound probes may not have a variable sequence (e.g., may have a target barcode but not a variable sequence). As such, in certain reaction schemes, variable sequences may be provided by unbound probes and/or by some but not all of the bound probes. In certain aspects, unbound probes and/or unbound reaction products or portions thereof may be amplified within the sample by primers in solution, as described further herein. Such amplification may spread a variable sequence (or collection of variable sequences on a reaction product) within a region (location) of the sample, such that the variable sequence (or collection of variable sequence) may act as a spatial barcode described herein (for example, similar to the reaction scheme shown by FIG. 21).

In certain aspects, bound probes may extend from unbound probes such that a longer bound reaction product is formed that can better reach (and hybridize to) surrounding bound probes (or other bound reaction products). For example, some or all of the unbound reaction probes may have the alternating complementary sequences (one member of each of the complementary sequence pair) such that bound probe extending along an unbound probe may end in a 3′ complementary sequence that can hybridize to and extend along other bound probes. Alternatively or in addition, some bound probes may have complementary sequences that hybridize to the complementary sequences of other bound probes (e.g., as shown in FIG. 10 or 11), and all or some of the bound probes may also extend along unbound probes.

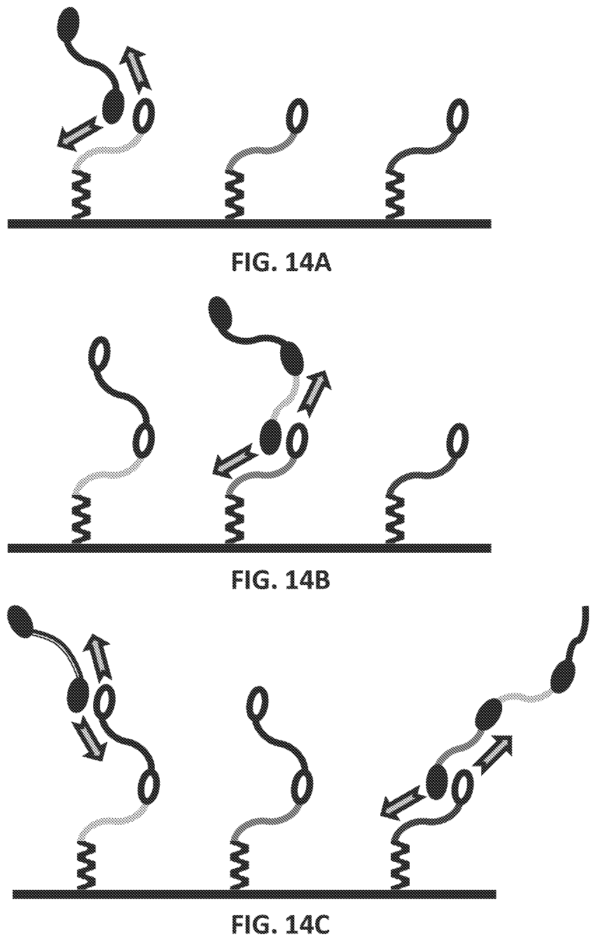

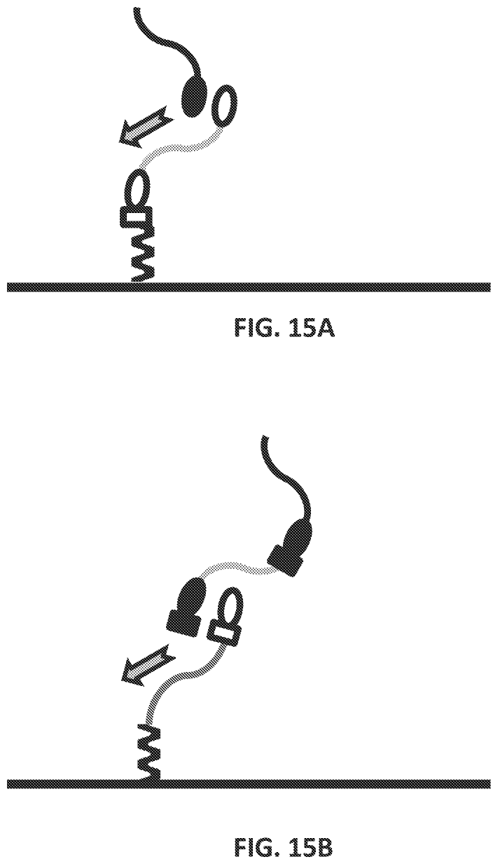

FIG. 15 provide an example of an ordered reaction scheme in which a first hybridization and extension of a first probe along a second probe ends in a 3′ complementary sequence comprising a different or additional sequence (e.g., as shown in FIG. 15A). For example, a first hybridization and extension may form a reaction product with the complementary sequence plus an additional sequence that together hybridize to other probe(s) with a higher melting temperature. Such a reaction scheme may bias reaction products to continue to react. Alternatively, a first hybridization and extension may form a reaction product with a shorter complementary sequence such that the reaction product may hybridize to other probe(s) at a lower melting temperature than the first hybridization, slowing down runaway reaction product formation and/or improving diversity of the reaction products. In certain aspects, the 3′ complementary sequence may be different from each of the complementary sequences of a first hybridization, such that the resulting reaction product can only react with a different probe (e.g., as shown in FIG. 15B). Such a probe may or may not have a 5′ complementary sequence. In certain aspects, the different probe may be an RNA specific probe. In certain aspects, the different probe may be from a set that binds a different target (or group of targets). The different probe may itself end in a new complementary sequence, or in one of the complementary sequences of the pair that formed the first hybridization.

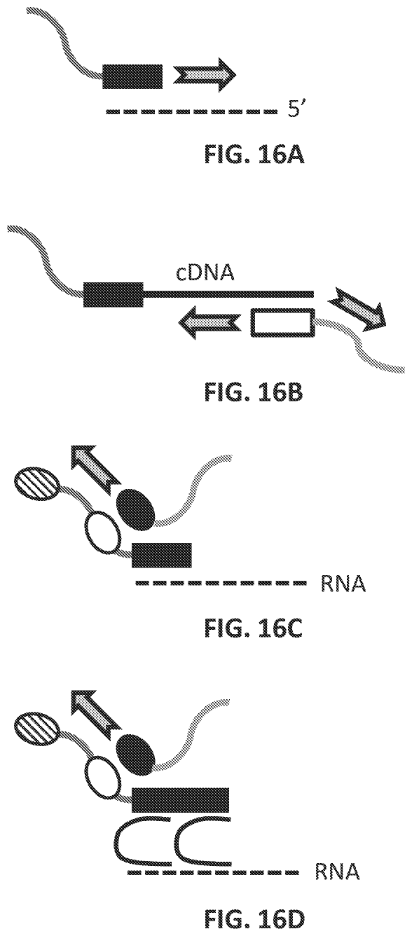

FIG. 16 provides a variety of reaction schemes for RNA detection. The RNA may be any RNA, such as mRNA. Alternatively, DNA (e.g., gDNA) may be targeted instead of RNA depicted in FIG. 16.

In the reaction scheme of FIG. 16A-B, an RNA specific probe first hybridizes to a target RNA (FIG. 16A). For example, the probe may hybridize to the poly-A tail, target (e.g., gene) specific sequence, or the probe may have a degenerate 3′ sequence that hybridizes to a random RNA sequence. An optional RNA degradation step may free resulting cDNA to be hybridized by another probe that extends for second strand synthesis. On or both probes may have variable sequences. One or both probes may instead be reaction products formed from an earlier extension step. The reaction product formed in FIG. 16B may end in a 3′ complementary sequence that can undergo additional hybridization and extension on other probes or reaction products.

In certain aspects, an RNA-specific probe may hybridize to an RNA and may provide complementary sequences on the 5′ side from the target hybridizing side. The complementary sequences may flank a variable sequence and/or a target barcode (e.g., identifying an RNA target the probe specifically hybridizes). Another probe my hybridize to a complementary sequence and extend along a portion of the RNA specific probe, as shown in FIG. 16C. The RNA-specific probe may not extend to form cDNA. Alternatively, the RNA-specific probe may extend to form cDNA, which may later be sequenced and its proximity related to other targets through the variable sequence (of the RNA-specific probe) that is incorporated into other reaction products.

In certain aspects, a hybridization scheme that improves specificity and/or provides signal amplification may use intermediate probes that hybridize to an RNA target and to a one or more probes, such as the example shown in FIG. 16D.

As shown in the above described figures, reaction schemes of the subject application (and related methods and kits) may include a set of oligonucleotide probes having a number of different elements. In many embodiments, a probe may include a target barcode that identifies an affinity reagent the probe is bound to. Such target barcode may be present alongside a variable sequence (e.g., both may be flanked by complementary sequences). A variable sequence may serve both to index a specific instance of a target bound by the probe with the variable sequence, and to determine spatial relationship of that instance of the target to instances of other targets in the sample (such as when the variable sequence is co-occurring with other variable sequences in a reaction product).

Although elements representing variable sequences (and optionally additional sequences) are in most or all of the probes of the above described figures, it will be appreciated that not all probes need to have variable sequences. For example, some probes may have target binding sequences and/or spatial barcodes but not variable sequences. Such probes may react with probes having variable sequences, such that reaction products have variable sequences sufficient to establish proximity of probes (or probe targets).

In certain aspects, the repeats of complementary sequences in proximity to their partner (the other complementary sequence of the pair) may allow for strand invasion by proximal probes or reaction products (and/or within a probe or reaction product) such that isothermal reactions take place. In certain aspects, isothermal reactions may be at lower temperatures than traditional PCR, such that binding of affinity reagents to targets and/or the integrity of the sample, is maintained.

In certain aspects, reaction products (or early reaction products) may be produced in live sample, e.g., such that cell movements are encoded.

### Reactions with Spatial Barcodes

Reaction schemes illustrated below provide examples of how nested spatial barcodes (e.g., “zip codes”) may be incorporated into reaction products. Such schemes could be combined or iterated to incorporate spatial barcodes on different scales. Zip code sequences may differentiate different occurrences of the same variable sequence present on separate oligonucleotide probes in different zip code regions. Reaction products comprising the same zip code(s) may be selectively amplified prior to sequencing, to further investigate a region of interest. Of note, spatial barcodes that cover different size regions and/or partially overlapping regions may be incorporated into the same reaction product.

Zip codes may be organized by known spatial locations (e.g., before or after reacting proximal oligonucleotide probes having different variable sequences). The oligonucleotides on the same particle may be synthesized to have a known spatial barcode (e.g., that corresponds to a known location at which the bead is applied to the sample). Alternatively, their spatial location can be related to one another based on zip code sequences shared by reaction products (i.e., as a relativistic spatial network). For example, particles may have oligonucleotides with a variable sequence formed by split-pool synthesis, such that the oligonucleotides on the same particle have the same variable sequence (the variable sequence only differs between particles). Such a variable sequence would act as a spatial barcode (e.g., zip code) as described herein.

In some reaction schemes, identical zip code sequences may be associated with the same particle (e.g., bead, polymer, droplet or long repeating oligonucleotide sequence). Alternatively, zip code sequences may be locally amplified. Identical zip code sequences associated with the same particle may be released (e.g., by cleavage) prior to incorporation into reaction products.

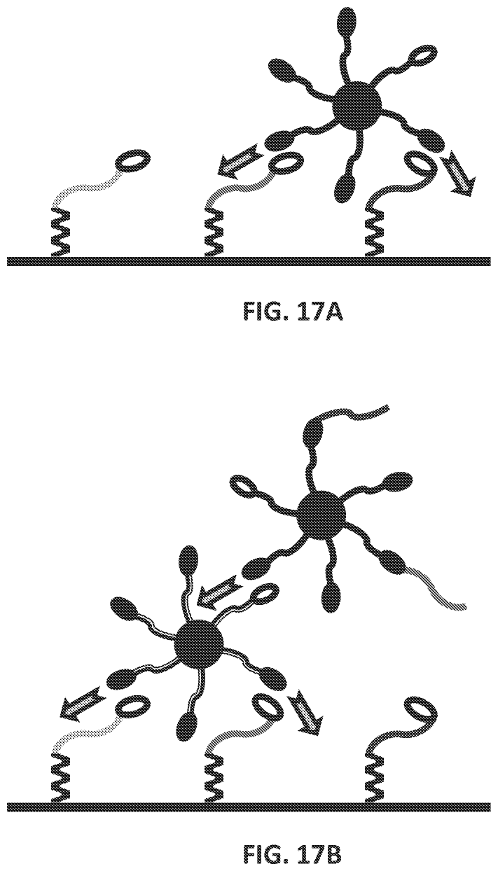

FIGS. 17 to 24 provides a variety of exemplary reaction schemes for nested spatial barcodes, in which a set of spatial barcodes are applied across a wider area of the sample (in some cases to predetermined locations). Reactions such as those shown in earlier figures may also occur (e.g., before, during, or after) incorporation of spatial barcodes into reaction products. As such, the probes shown in FIGS. 17 to 24 (or in any spatial barcode reaction) may be reaction products that incorporate spatial barcodes.

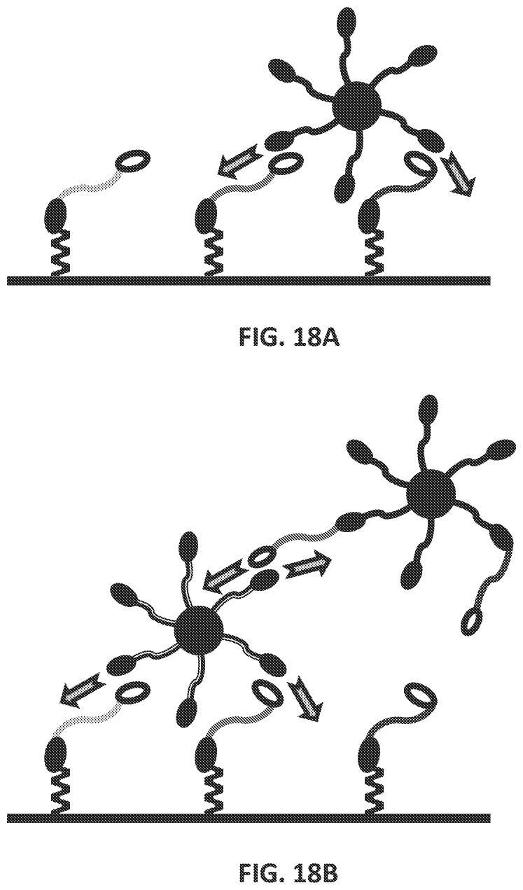

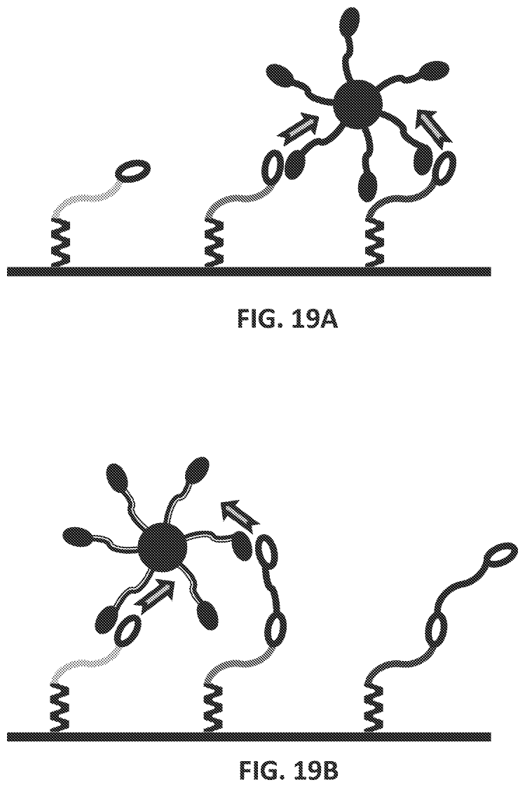

FIGS. 17 to 19 show a particle having a plurality of separate oligonucleotide probe sequences that have the same variable sequence (or same spatial barcode). As shown in FIGS. 17 and 18, some oligonucleotides one separate particles may hybridize and extend along one another (unidirectionally or bidirectionally) such that some of the reaction products formed relate the proximity of spatial barcodes to each other. Some or all of the oligonucleotides on the particles may extend along bound probes. As shown in FIG. 19, bound probes may extend along oligonucleotides of particles, forming reaction products that incorporate spatial barcodes (e.g. variable sequences conserved across oligonucleotides of a particle). While FIGS. 17 to 19 show exemplary reaction schemes, it will be appreciated that combinations of the elements across these Figures may also provide suitable reaction products.

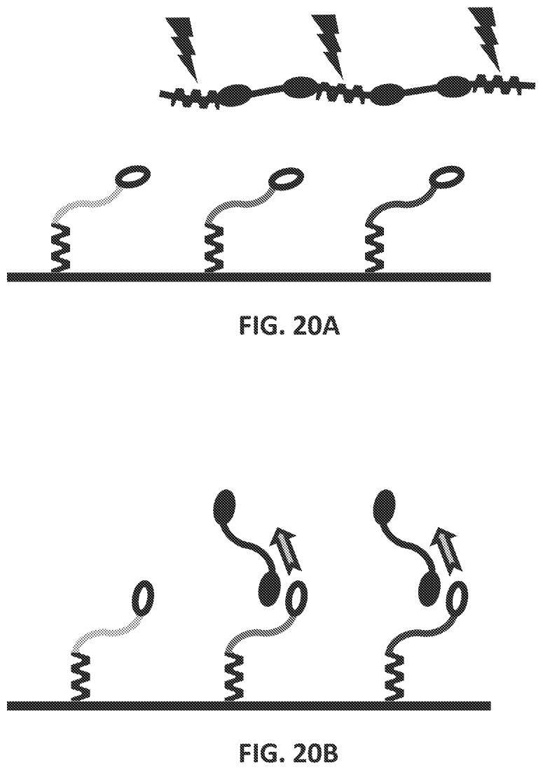

In the exemplary reaction scheme of FIG. 20, a particle comprising a plurality of probes with the same variable sequence (e.g., spatial barcode) may be cleaved. The particle may be linear or branched. The cleavage may occur at nucleotide sequences or linkers between probes. The cleaved probes may then distribute in the sample at a particular location (or region) around where the particle was cleaved, and may form reaction products with bound probes. In this example, bound probes are shown extending along the cleaved probes, although it will be understood that extension could occur bidirectionally or in the other direction (e.g., in an alternative reaction scheme) and form suitable reaction products.

In certain aspects, unbound probes and/or unbound reaction products may be amplified. In the exemplary reaction scheme of FIG. 21, an unbound probe may be amplified to product multiple copies of the unbound probe as shown in FIG. 21A and 21B. An unbound probe may form unbound reaction products with bound probe (e.g., in a unidirectional extension shown in FIG. 21C or in a bidirectional extension). Such reaction products may themselves be amplified, for example, as shown in FIG. 21D. Amplified unbound reaction products may go on to react with additional bound probes and/or bound reaction products.

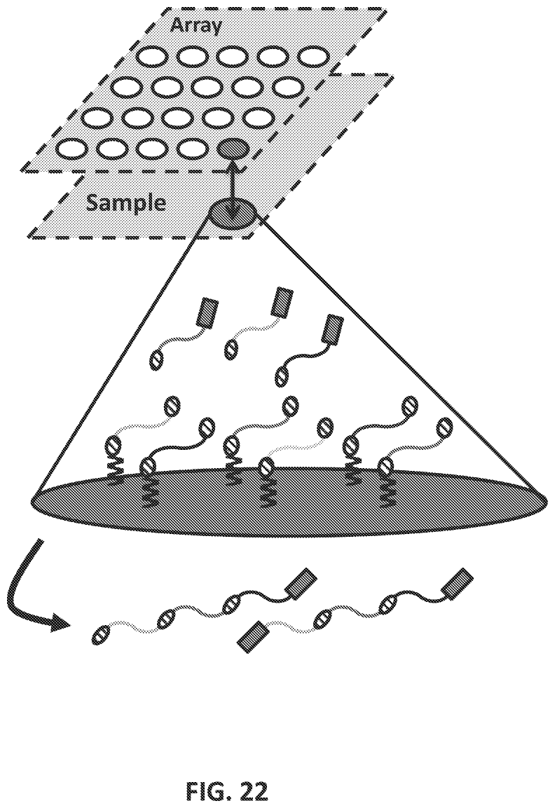

FIGS. 22 to 24 provide physical means by which spatial barcodes may be addressed.

In FIG. 22, an array of oligonucleotide probes have predetermined spatial barcodes associated with their position in the array. The spatially barcoded probes are applied to a sample (e.g., having bound probes). Reaction products may be formed incorporating bound probe sequences (e.g., having variable sequences and/or target barcodes) and spatial barcodes.

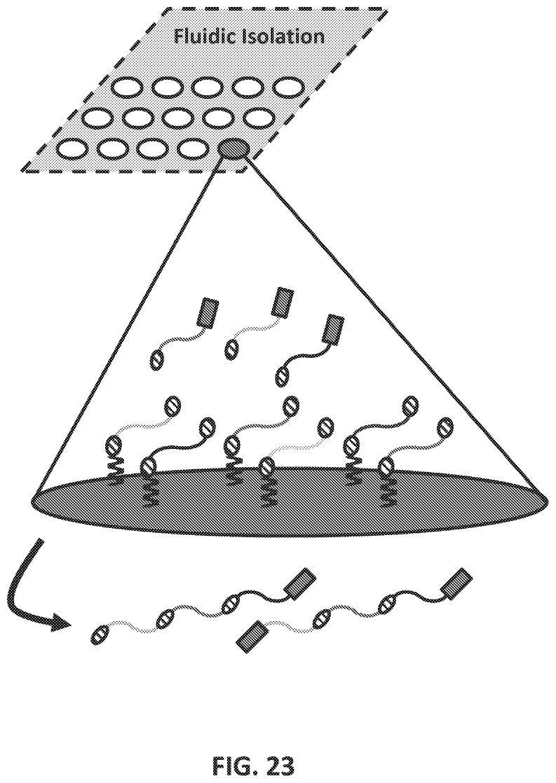

In FIG. 23, portions of a sample are fluidically isolated (e.g., in wells, fluidic chambers, or droplets) and barcodes are applied. The location of the sample portions may be predetermined (e.g., may be excised from known locations of a tissue slide) and may be associated with a spatial barcode after fluidic isolation, or the location of the sample portions may be determined from sequencing reaction products.

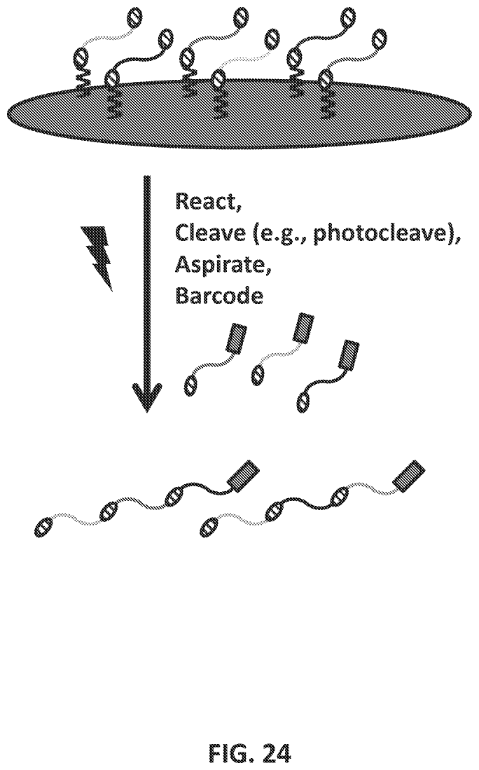

In FIG. 24, oligonucleotide probes are reacted in sample and reaction products are removed (e.g., may be cleaved, such as photocleavage of a linker), and the released reaction products are reacted with spatially barcoded probes. The release of reaction products may be location specific (such as photocleavage with a laser) and the spatial barcodes may be of predetermined sequence and associated with the location. This allows sequencing of reaction products with the spatial barcodes to identify the location (region) of the sample that the reaction product (and its encoded probes) came from.

### Enrichment of Reaction Products

Certain targets, combinations of targets, and/or regions of interest may be enriched prior to sequencing of reaction products.

For example, complimentary sequences may enrich for formation of reaction products encoding a combination of protein targets, RNA targets, and/or zip code sequences. Alternatively or in addition, an initial extension may result in a longer complementary sequence that increases the rate of successive extensions (e.g., to induce subsampling).

Some oligonucleotide probes and/or their extension products may allow for RNA detection by hybridization and potentially extension. Some probes and/or their extension products may hybridize to a target specific RNA sequence, poly-A tail, and/or random sequence. Reaction products encoding RNA targets may be formed by extension, tailing, template switching, ligation and/or other techniques known in the art.

Reaction products may have sequences that can be primed and amplified to enrich for specific targets and/or regions. For example, primer(s) specific for one or more zip code(s) may be used to amplify reaction products comprising those zip code(s). Alternatively or in addition, primer(s) specific for a one or more target (or their identifiers) may be used to amplify reaction products encoding those target(s). As such, the same set of reaction products may be sequenced and investigated for different targets and/or regions.

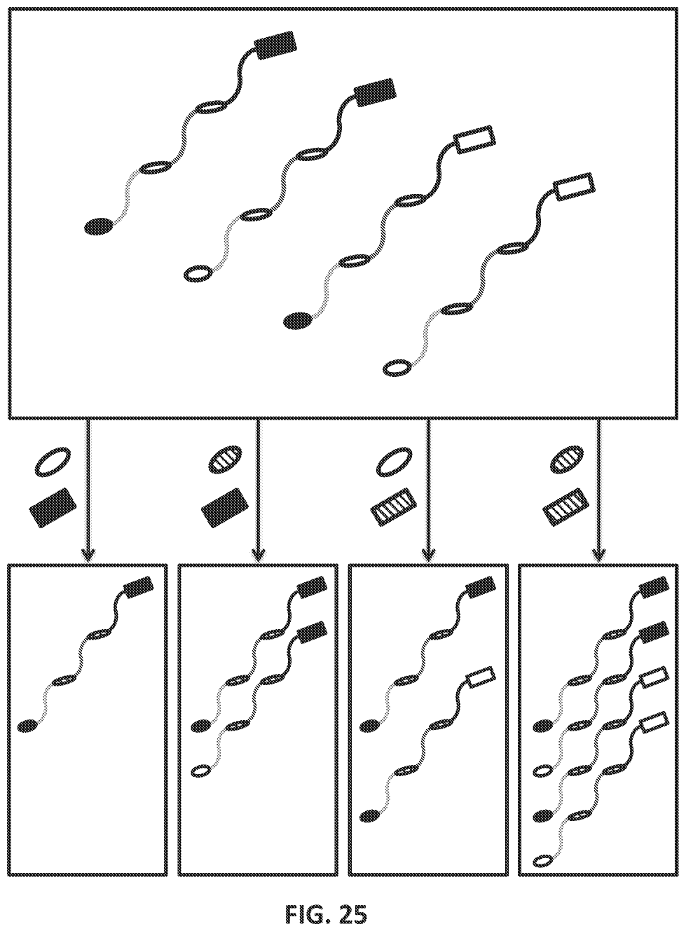

FIG. 25 provides exemplary reaction schemes in which certain reaction products are enriched prior to sequencing. Reaction products are shown having different sequences at each end (top). The primers (middle) used to amplify reaction products (e.g., prior to sequencing) may selectively amplify a subset of the reaction products (bottom). Two or more primers may amplify specific spatial barcodes and/or target barcodes, such that one or more regions and/or targets of the sample are enriched. Some primers may hybridize and extend from a plurality of spatial barcodes or a plurality of target barcodes. A pool of reaction products may be reinvestigated for different regions and/or targets using this method.

Enrichment may include binding of desired spatial barcode(s) and/or target barcode(s) of reaction products to a solid surface, and removal of unbound reaction products. For example, enrichment probes that specifically hybridize to desired spatial barcode(s) and/or target barcode(s) (and/or their revers complements) may be mixed with reaction products. Enrichment probes may then be pulled out of solution, such as through binding to beads (e.g., through binding of biotin group on the enrichment probe to streptavidin presented by the beads). In another example, desired spatial barcode(s) and/or target barcode(s) of reaction probes may be hybridized directly to beads that present complementary oligonucleotides.

### IV. Sequencing

Reaction products may be provided or obtained by the methods described above. The reaction products may be prepared for sequencing (e.g., as described above).

Sequencing of the reaction products may be by any means known to one of skill in the art. For example, sequencing may be by single molecule sequencing. Alternatively, sequencing may be of clonal clusters (e.g., produced by bridge amplification). Sequencing may be through a sequencing by synthesis method, such as when a primer is extended along the reaction product to be sequenced. Sequencing may be obtained by a fluorescence readout. Alternatively, sequencing may be by a direct electronic readout.

### V. Analysis of Spatial Relationships

As described herein, sequencing data from reaction products may be used to analyze spatial relationships of probes (e.g., probe targets). In certain aspects, the spatial relationship may be the relative proximity of targets. The analysis may include a metric or score of proximity between pairs of targets. The analysis may be of the correlation or effect of a microenvironment (e.g., presences of one or more targets) on the abundance or colocalization of one or more targets.

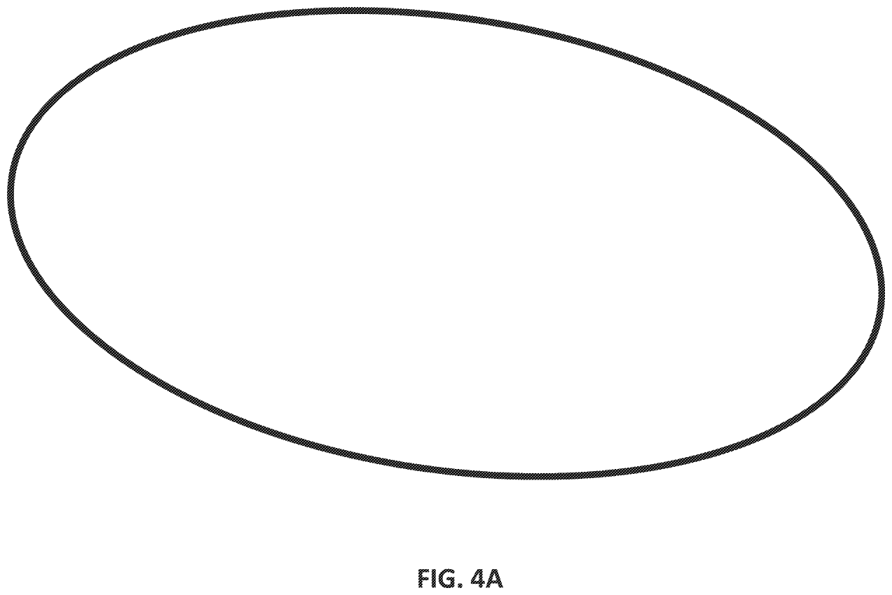

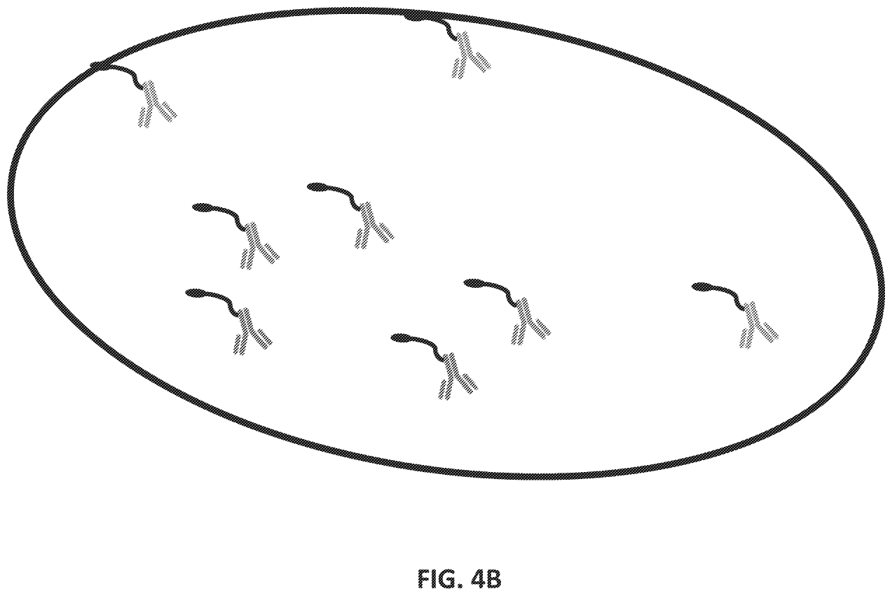

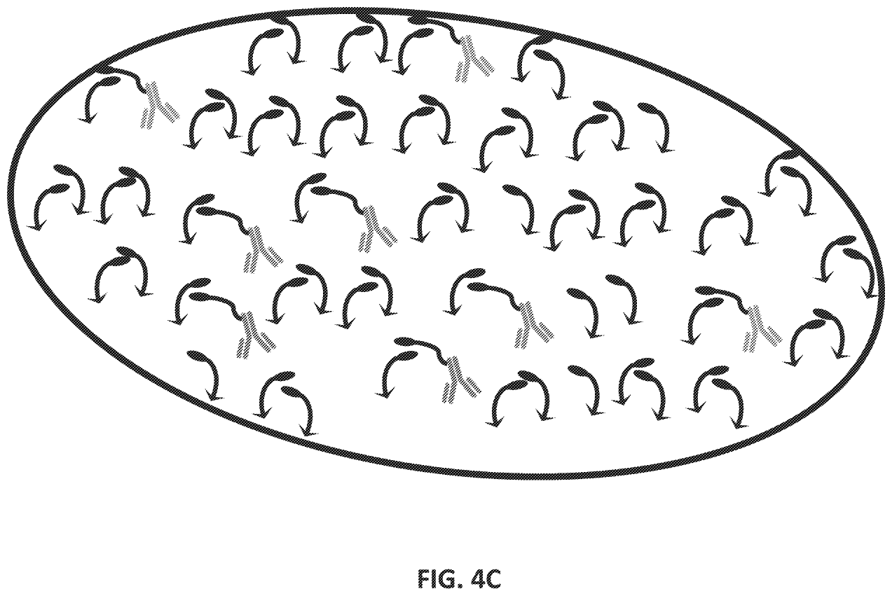

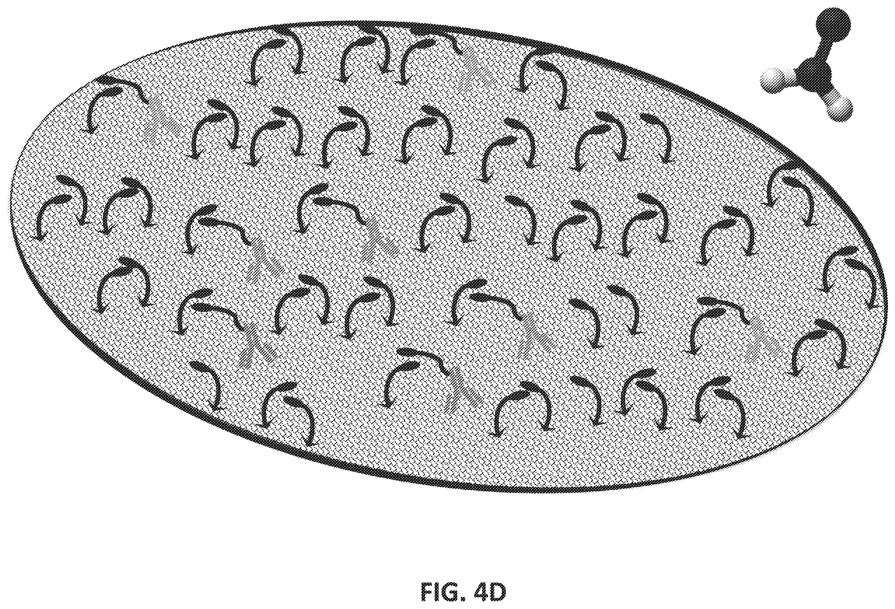

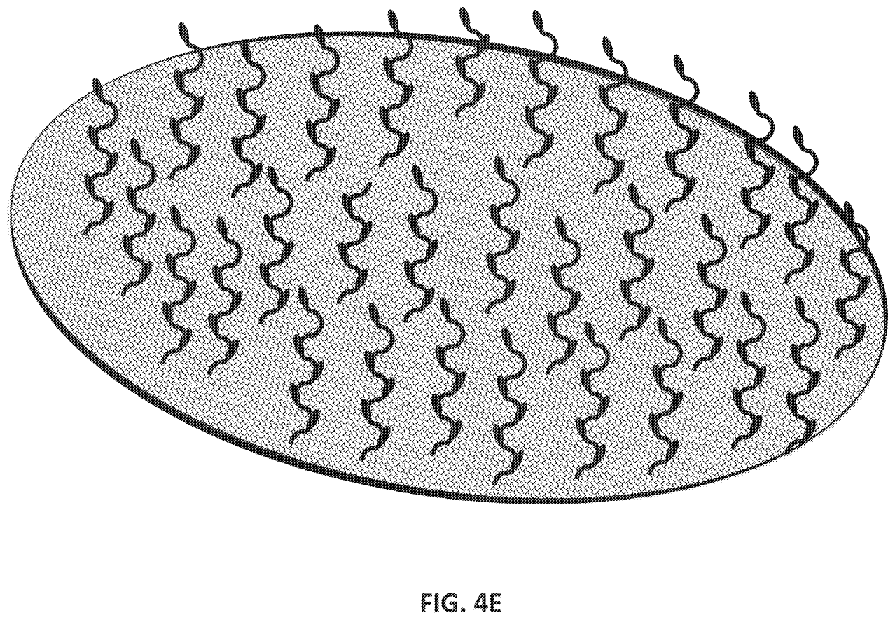

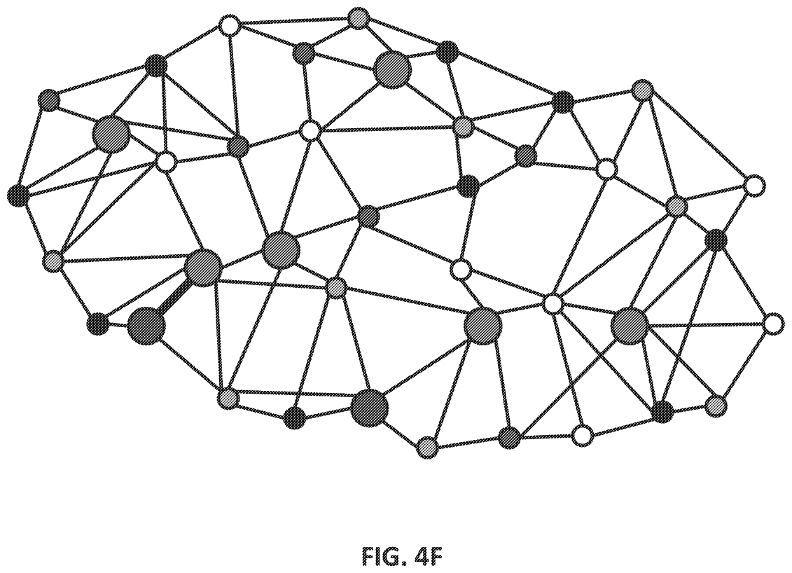

In certain aspects, an analysis may include a visual representation of the relative spatial relationships of probes. Such a visual representation may be a graph, or image based on a graph. The graph may include nodes that represent at least one instance of a probe and edges between nodes represent instances where the two probes were encoded in the same reaction product (e.g., when the probes were immobilized in proximity in the sample). Such a graph may not have an external frame of reference, but may represent spatial relationships of individual probes, including of probes that are not both encoded on the same reaction product (e.g., second, third order relationships). For example, a reaction products formed in a cell or tissue (e.g., as shown in FIG. 4) may be sequenced and spatial relationships represented as a graph (e.g., as shown in FIG. 4F).

Depending on the number of probes and length of the variable sequence, the same variable sequence may occur on different probes (e.g., when there are more probes than variable sequence). In such cases, barcodes on the probes (or on reaction products formed from probes) may differentiate variable sequences. For example, two probes with different target barcodes but the same variable sequence can be differentiated, as a variable sequence and target barcode from the same probe may be incorporated into the reaction product such that they are identified as coming from the same probe.

Spatial barcodes may also be useful to differentiate identical variable sequences that come from different probes. For example, if the same variable sequence (or combination of a variable sequence associated with a target barcode) is in two reaction products with different (e.g., distant) spatial barcodes, the variable sequence may be identified as coming from different probes (e.g., indicating a different instance of a target).

For example, a variable sequence 10 nucleotides long may provide 4L10 (around 1 million) different sequences. However, more than 1 million probes may be reacted to form reaction products that are sequenced. If probes in a set have 100 different target barcodes, then there are 100 million distinct probes (variable sequences*target barcode sequences). Use of spatial barcodes, length barcodes, or any other barcodes may also increase the number of probes that can be distinguished.

Polymerase error, sequencing error, redundant variable sequences (e.g., probe sequences having the same variable sequence and optionally additional identical barcode(s)) and/or floating probes (unbound probes that were expected to be bound) may confuse the readout of spatial relationships of probes (e.g. or their targets).

For example, the incorrect read of a variable sequence may populate a new (and incorrect) instance of a probe or its target, such as a new node in a graph described herein. As such, variable sequences (or probes/targets represented by a variable sequence) that occurs in one (or, e.g., less than 2, less than 3, less than 4) instances may be removed from the data set.

In another example, a redundant variable sequence, or an unbound probe that was expected to be bound, may lead to false associations between probes or their targets (e.g., edges between nodes that do not reflect correct proximity of probe/targets). This issue may be resolved or reduced by removal of sparse connections (e.g., such that the graph only shows well connected nodes that have better representation of their spatial relationship to other nodes).

As such, probes (e.g., nodes in a graph) and their associations (e.g., edges in a graph) may be pruned when sparse to reduce artifacts and/or improve the certainty of the spatial relationships of the remaining nodes. In certain aspects, several indirect connections (a larger incidence of short paths) between nodes may indicate the nodes are correctly related. Where a single instance of a probe (e.g., variable sequence) is read as being in close spatial proximity to two or more distal parts of the sample (e.g., graph representing the sample), the probe (or node representing the probe) may be split into multiple probes (or nodes). This may be useful when the number of probes read is greater than the expected number of distinct probes (e.g., variable sequences). Longer variable sequences would reduce multiple instances of the same probe (or variable sequence).

In certain aspects, a graph or other representation of spatial proximity based on the sequencing of reaction products formed by the subject methods, may be superimposed on a visual image (e.g., microscopy image) of the sample. Spatial barcodes may be used for such a superimposition.

### Nested Spatial Barcodes

Aspects of the invention may include applying a first set of spatial barcodes to the sample before disaggregating the sample, and optionally applying a second set spatial barcodes to the sample. The first spatial barcode may identify the location of disaggregated sample prior to the disaggregation, and may be an objective or relativistic barcode. For example, a spatially encoded array of probes may be applied to a solid biological sample, after which cells or tissue fragments of the sample are disaggregated. For example, a fresh, frozen or fixed tissue sample may be disaggregated by one or more of trypsinization, laser dissection microscopy, agitation, sonication, grinding of tissue through a filter, application of certain buffers and/or proteinase treatment.

For example, a method of spatially encoded single cell analysis may include i) applying spatially barcoded probes to a sample, wherein the probes have an known location (e.g., are on spots in an array), ii) disaggregating particles in the sample, and iii) associating protein and/or RNA targets with the spatial barcode. The particles may be cells. Step iii) may include isolating the particles (e.g., isolating cells in droplets) alongside barcoded beads, and reacting the bead barcode with targets (e.g., through direct hybridization to a target RNA and/or by hybridization to target specific probes) and with a spatial barcode (e.g., on the same or different reaction product). For example, a spatial array of barcoded oligonucleotide probes may be applied to a tissue section, and the probes may be bound covalently to the cells (e.g., by amine reactive chemistry such as through NHS ester, thiol reactive chemistry such as through maleimide, or by crosslinking such as formalin fixation of a lysine tail on the probe with lysines on the cell). Cells may then be disaggregated, and optionally stained with oligonucleotide-tagged antibodies. Cells may then be isolated in droplets alongside beads comprising oligonucleotides with a bead barcode (e.g., barcode specific for probes on the bead). The isolated cells may be lysed to release targets such as RNA, DNA and/or proteins (or oligonucleotide-tagged antibodies bound to target proteins). The oligonucleotides on the bead may comprise a target binding sequence, such as a poly-T 3′ sequence that hybridizes and extends from the poly-A tail of RNA, thereby reverse transcribing the RNA into a cDNA sequence associated with the bead barcode. The oligonucleotides on the bead may comprise additional sequences for sequencing library preparation. If the cells were stained with oligonucleotide-tagged antibodies, the oligonucleotide-tag may comprise a poly-A sequence (or another sequence for reaction with the oligonucleotide probes on the bead) and a target ID (specific for the target). Reaction products that together encode one or more spatial barcodes and target IDs with the same bead barcode can be used to identify the location of the cell and its associated targets) in the tissue prior to disaggregation. After reaction with the bead barcoded oligonucleotides, reaction products from different isolated cells may be pooled and prepared for sequencing (e.g., by one or more of reaction product purification, preamplification, targeted amplification/enrichment, and adaptor incorporation prior to or after pooling). In some cases, a combination of spatial barcodes could be used to identify the location of a cell that is between two or more spots in the array, and may thereby provide better resolution than the size of spots in the array. In some cases, less than 50%, less than 20%, or less than 10% of the disaggregated cells are analyzed, which may allow for cost reduction through downsampling. Isolating cells may allow detection of single cells, where application of the spatially arrayed probes by themselves would not allow single cell resolution. The lysis of isolated cells may improve sensitivity through improved release of targets for detection and/or kinetics for reaction product formation, compared to running a reaction in intact tissue or on a solid surface such as an array.

### Applications and Utility

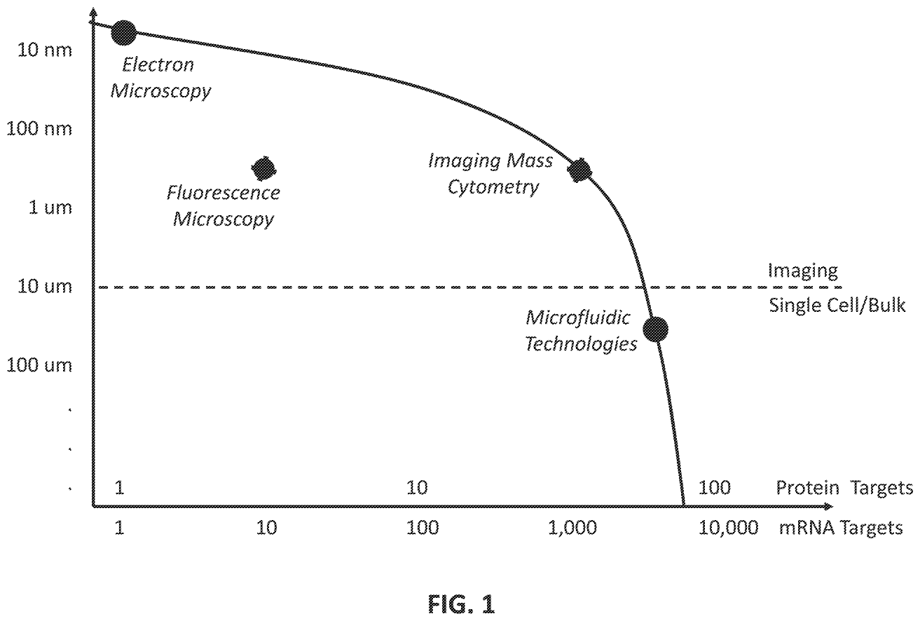

As shown in FIG. 1, many technologies that enable multiplexed detection exhibit a trade-off between plexity and spatial resolution.

The subject methods and kits may allow flexible imaging of cells and/or tissue across different resolutions (single molecule through single cell) using same technology. In some workflows, spatial network at a lower resolution may have specific areas at higher resolution (e.g., cell-cell interactions represented at high resolution). Imaging of multicellular intact tissue may be performed in tube by the subject methods (e.g., in suspension) as oppose to on a slide.

Spatial profiling of microenvironment of small group of cells (e.g., isolated and/or cultured together) has applications in understanding cancer, immune infiltrated tissue, developmental biology (e.g., of blastocyte), and stem cell biology. For example, multiplexed spatial profiling of individual cells at subcellular and/or single molecule resolution (e.g., less than 100 or less than 50 nm) has applications in stem cell biology (e.g., to assay the complex asymmetric localization of proteins and/or RNA to create 1 differentiated daughter cell and 1 stem daughter cell), in signaling networks (e.g., one can rely on local perturbations/fluctuations in targets and their interactions in different regions of a cell, each region being its own sub experiment), and in identification of protein clusters and their spatial relationships (complex protein structures in cell, protein islands on cell surface) such as in cell-cell contacts that drive immune response.

Any of above could be combined with treatments to screen for effect of treatment (e.g., screen for disruption of target expression or association with other targets) or identify secondary drug targets for combination therapy (e.g., secondary drug target may be a target with a change in abundance and/or association with other targets). Such treatments include small molecule, gene editing, and antibody based treatments.

### Spatial Barcoding prior to Cell Dissociation

Aspects of the subject application include a method of identifying spatial relationships within a sample may include identifying the spatial relationship of cells in the sample (e.g., with or without further identifying the spatial relationships of probes or targets in the sample described above). Such a method may include applying spatial barcodes (spatial barcode oligonucleotide sequences, such from spatial barcode probes) to different locations in a tissue sample, wherein instances of a same spatial barcode sequence are applied to a plurality of cells within the same location. Further, a cell at a location may be associated with multiple different spatial barcodes (e.g., spatial barcodes that were applied to an overlapping or partially overlapping location, such as when a spatial barcode is applied directly to a proximal location and then diffuses into the location of the cell). As such, the combination and amounts of unique spatial barcodes associated with (on or in) a cell may together identify that cells location in the sample (e.g., compared to an objective frame of reference such as an optical image or coordinate, and/or relative to other cells in the sample). In certain aspects, the majority of individual cells are each associated with a plurality of different spatial barcode sequences.

After application of the spatial barcode, cells may be dissociated from the tissue sample. A variety of enzymatic (e.g., trypsin based) and physical means of dissociation are known in the art, and any suitable means of dissociation may be employed provided it leaves desired targets (such as protein and/or RNA of interest) intact. When DNA targets are of interest, nuclei may be dissociated and processed in place of the cells themselves.

Individual dissociated cells with a cell barcode sequence, such as in a fluidic chamber or in a droplet. The cell may be lysed. Lysis may release contents (such as RNA, spatial barcodes, and/or target barcodes) into solution, which allows for efficient reaction with cell barcode sequences to produce reaction products.

Aspects include incorporating spatial barcode sequences of an individual cell and a cell barcode sequence into reaction products, such that a reaction product comprises both a spatial barcode sequence and the cell barcode sequence. The same reaction product, or additional reaction products, may incorporate cDNA with the cell barcode and/or may incorporate target barcode sequence (e.g., of a target barcode probe) with the cell barcode.

Sequencing of the above reaction products (e.g., after further library prep and/or amplification) can be performed. Spatial relationships of individual cells (prior to dissociation) may be determined based on the sequencing of reaction products. For example, cells that share at least some spatial barcode sequences may be closer to one another than cells that do not share spatial barcode sequences. Predetermined (known) locations of spatial barcode sequences applied to the sample may be related to coordinates, such that cell events can be assigned a coordinate based on the spatial barcode sequences indexed with the cell barcode that the cell was isolated with. Alternatively, the locations of spatial barcode sequences applied to the sample may be determined based on coincidence in and between cells, such that the locations of cells are determined relative to one another. As such, the locations the spatial barcode sequences are applied to are known prior to sequencing, or mare not known prior to sequencing.

The method may further comprising incorporating cDNA of the individual cell with the cell barcode sequence in step d) to form individual reaction products that comprise the cell barcode sequence and a cDNA sequence (e.g., or its reverse complement).

The method may include applying target-specific oligonucleotide probes to disaggregated cells before step c) of isolating, wherein the target-specific oligonucleotide probes comprise an antibody and a target barcode sequence. The method may further include incorporating the target barcode sequence with the cell barcode sequence in a reaction product.

The method may include selecting one or more populations of dissociated cells for isolation and indexing. For example, cell populations may be selected is by fluorescence activated cell sorting. Cells or cell populations may be downsampled, for example to reduce cost and/or when the cell populations are of less interest, are homogenous, and/or are overabundant compared to other cell populations of interest.

In certain aspects, the tissue sample is a tissue section thicker than 10 microns, such as a section thicker than 20 microns or thicker than 40 microns. A thick tissue section may include more whole cells that would remain intact after dissociation.

In certain aspects, the spatial barcode sequences may applied to the tissue sample as spatial barcode probes comprising the spatial barcode sequence, such as from a solid support (e.g., a planar array or beads). For example, spatial barcode probes may be applied as an array, and wherein individual spatial barcode sequences occupy a known location on the array. Release of spatial barcode probes from a solid support may be by wetting of spotted spatial barcode probes, chemical cleavage, enzymatic cleavage, photolytic cleavage, thermolytic cleavage, or dehybridization from a complementary probe immobilized on the solid support.

The spatial barcode probes of the array may be released by wetting, photolytic or chemical cleavage, by dehybridization to a complement covalently bound to the array, or any suitable means. Spatial barcode probes of an array may be further applied to the tissue sample by direct contact (such as applied pressure to the tissue sample). In certain aspects, provides a spatial resolution at which the location of the cell is determined (e.g., with more than 50% confidence) is better (smaller) than the spot size of the array.

In certain aspects, spatial barcode probes are applied to the tissue sample as beads, wherein spatial barcode probes of the bead comprise the same spatial barcode sequence.

The method may further include binding the spatial barcode probes to cells of the tissue sample in a diffusion limited manner, e.g., such that the majority of spatial barcode probes are bound within a specific location of the sample. To bind in a diffusion limited manner, the rate of binding may be sufficiently fast. For example, the majority of probes that are bound to the sample may be bound within minutes or seconds, such as less than 30 minutes, 10 minutes, 5 minutes, 2 minutes, 1 minute, or 30 seconds. The time it takes the probes to bind (or the time the probes are allowed to bind the sample before unbound probes are inactivated and/or washed off) may be less than the time it takes for the probe to diffuse through the sample, such as the majority of probes diffusing less than 10 mm, 5 mm, 1 mm, or 100 urn of the sample.

In certain aspects, the spatial barcode probes comprise a bulky group that limits diffusion. Alternatively or in addition, lateral diffusion may be reduces such as through a gel positioned between spatial barcode probes and the tissue. Application of expansion gel to the tissue may expand the tissue and provide a matrix that standardizes diffusion rate across the tissue. The method by which probes bind the sample may also limit the diffusion rate of the probe, such as lipophilic probes or probes that hybridize to complementary sequences patterned across the tissue sample.

In certain aspect, spatial barcode probes are functionalized for binding to the sample, such as to the cell surface. For example, spatial barcode probes are functionalized with a thiol-reactive group that binds to thiol groups naturally present on the cell surface, such as an acrydide or maleimide group. In certain aspects, spatial barcode probes comprises a lipid group that incorporates into the cell membrane. The sample barcode probe comprises a group that initiates cell uptake, such as a phosphate group or peptide that mediates cell uptake. In any of these schemes, the cells may be alive.

Aspects include pre-treating the tissue sample to provide attachment sites for the spatial barcode probes. Such an attachment site may be a reactive functional group the spatial barcode probe specifically reacts with (such as by click chemistry, such as strain-promoted click chemistry), an oligonucleotide the spatial barcode probe specifically hybridizes to (such as an antibody bound oligonucleotide that binds an analyte on the cell surface), or an affinity target the spatial barcode probe specifically binds to (e.g., such as a biotin-streptavidin binding). In certain aspects, the spatial barcode probe is bound through cross-linking after application to the tissue sample, such as by addition of formaldehyde to sample after application of amine (e.g., lysine) functionalized spatial barcode probes.

In certain aspects, spatial barcode sequences are associated with the cells through extension of immobilized probes by a polymerase. For example, spatial barcodes can be incorporated into reaction products by any of the means discussed in this application. Such extension may be under isothermal conditions, such as via strand displacing hairpin (e.g., the spatial barcode probe may be a strand displacing hairpin that acts as a template for a plurality of immobilized oligonucleotides in the location). Strand displacing hairpins include designs used in the SABER method, in which extension along the hairpin starts and ends with a repeat sequence, and wherein the hairpin comprises a complementary sequence that competes for hybridization to the repeat sequence. In certain aspects, extension is a successive extension as described herein.

As described earlier, a cell barcode can be incorporated into reaction products with one or more spatial barcode sequence, cDNA sequence, gDNA sequence, and/or target barcode sequence (e.g., in a single reaction product or in separate reaction products). The reaction products may be sequenced and the spatial barcodes along with any cDNA, gDNA and/or protein targets may be identified with the same cell barcode. Targets (e.g., protein or RNA targets) may be used to identify the cell type and/or function. In certain aspects, targets (e.g., target barcodes indicating presence of a specific protein and/or specific genes) may be selectively amplified or otherwise enriched prior to sequencing.

The location of cells may be identified and rendered based on the sequencing described earlier. Specifically, the combination and amounts of distinct spatial barcodes sequences associated with a cell (e.g., indexed with the same cell barcode) may identify the coordinates of the cell and/or proximity to other cells sharing at least some of the spatial barcode sequences. An image of cells in the tissue may be provided, or a proximity score between cells and/or cell types may be provided.

Methods may further include generating an image integrating cell location and cell expression of RNA and/or protein targets based on the sequencing. The image may be co registered with an optical image (such as a brightfield or fluorescence microscopy image) of the tissue sample obtained before dissociating the cells. In certain aspects, the image may be a graph in which nodes represent individual cells or groups of cells. Cells (or nodes representing cells) may be colorized to represent cell type and/or function identified based on a plurality of protein and/or RNA targets. The image (or rendering) may be interactive, such that a user can define cell types (or expression profiles) of interest and visualize or quantify their distribution. Aspects may include generating an 3D image of cell location, such as when the location of spatial barcode sequences applied to the sample are distinct in 3 dimensions.

In certain aspects, a method of single cell sequencing with spatial resolution may include applying a spatial barcode array to cells in a tissue sample, dissociating cells from the tissue sample, isolating individual dissociated cells in droplets with a bead comprising a cell barcode, lysing isolated cells, incorporating spatial barcodes of an isolated cell with a cell barcode to form individual reaction products that comprise the cell barcode and a spatial barcode, incorporating the cDNA of the isolated cell with the cell barcode to form individual reaction products that comprise the cell barcode and a cDNA sequence, and sequencing the reaction products. The method may further include staining dissociated cells with target specific probes comprising an antibody and target barcode prior to isolating cells, incorporating target barcodes with the cell barcode to form individual reaction products that comprise both a target barcode and a cell barcode sequence. Single cell expression of protein and/or RNA targets may be rendered along with the location of the single cells, based on the sequencing of the reaction products. Aspects include a kit comprising a plurality of the sample barcode probes of any one of the methods described above. For example, sample barcode probes organized on a support and functionalized for attachment to cells in a sample, optionally along with other reagents for binding the sample barcode probes to the sample and/or forming reaction products, may be provided in a kit.

Alternatively or in addition, fragments of tissue (as opposed to single cells) may be dissociated and processed as described in any of the above methods. Further, while a tissue is described in the embodiments above, such embodiments may be modified for any cellular sample in which the location of the cells are of import.

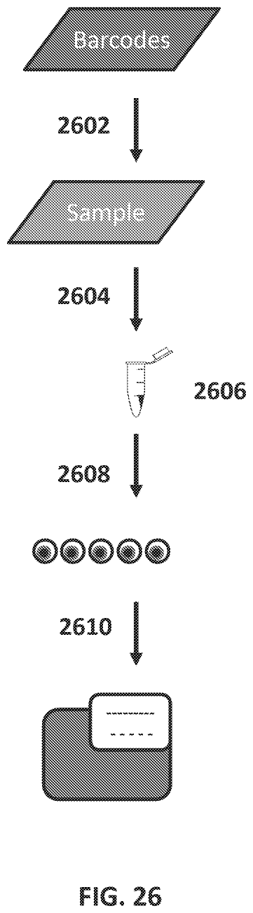

FIG. 26 provides a sequencing workflow for identifying locations of cells in a tissue sample. In step 2602, spatial barcode sequences (e.g., oligonucleotide probes comprising a spatial barcode) are associated with a sample, such as a tissue sample, as described further herein. The tissue sample may be thick enough to have whole cells (e.g., may be at least 10 microns, 20 microns, or 50 microns thick). The tissue sample may be a section of tissue, such as from a needle biopsy or excision. In step 2604, sample is dissociated into individual cells, such as can be done with the Miltenyi gentleMACS dissociator. In step 2606, cells may optionally be processed, such as by enrichment of one or more cell populations (e.g., by FACS) and/or staining with antibody-oligo probes (or other target-specific probes described herein). In step 2608, cells are isolated such as in droplets, microwells, or fluidic chambers. A cell barcode may be associated with individual isolated cells, such as by co-encapsulation of a bead with a cell in a droplet or application of sample barcoded primers to volumes (e.g., microwells or fluidic chambers) with isolated cells. Cells may be lysed, optionally reverse transcribed, and indexed with cell barcode (such that spatial barcodes and optionally cDNA is incorporated into reaction products with the cell barcode). In addition to single cell indexing of spatial barcodes, the RNA, DNA and/or protein targets may be indexed with the same cell barcode as described further herein. In step 2610, library prep and sequencing allows identification of single cell expression (e.g., RNA and/or protein expression) with cell location (e.g., represented by the indexed spatial barcodes). In certain aspects, a cell may have a plurality of different spatial barcodes that together approximate the location of the cell. Cell location can be rendered as discussed further herein, and may optionally be coregistered with cell expression and/or an optical image taken of the tissue sample before disaggregation.

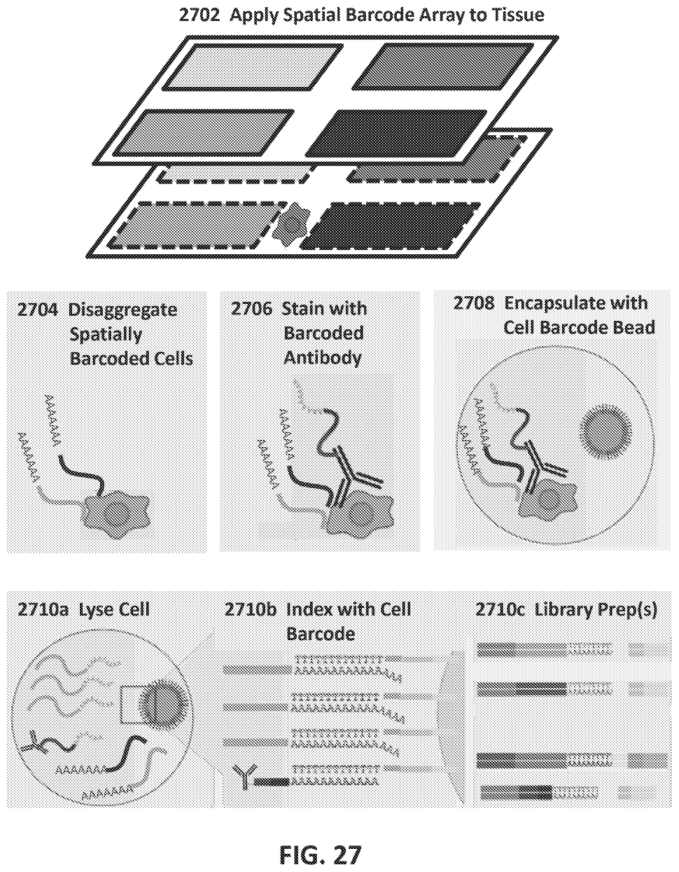

FIG. 27 provides an example of the FIG. 26 workflow in which a spatial barcode array is applied to a sample. In step 2702, an array (top) of spatial barcode probes is applied to a tissue sample (bottom). The spatial barcode sequence of individual clusters/locations may be known (e.g., recorded during printing of the array, or determined by sequencing on the array). The four shaded quadrants each represent a cluster of spatial barcode probes comprising the same spatial barcode. The dashed border around the quadrants imposed on the sample represent the diffusion of the spatial barcode probes upon application to the sample. A cell is shown between the lower two quadrants. In step 2704, the sample is disaggregated into spatially barcoded cells. The cell from the sample is now shown with sample barcode probe from each of the proximal clusters of spatial barcode probes. Of note, individual cells may have at least 1, at least 2, at least 5, or at least 10 different spatial barcode probes (having different spatial barcode sequences). The combination and relative number of spatial barcode probes from different clusters can be used to identify the location of the cell (e.g., with higher accuracy than the size of the diffused cluster, or even the size of the cluster when organized on the array). In step 2706, the cell is processed (stained with an antibody conjugated to a target barcoded oligonucleotide). In step 2708, the cell is encapsulated with a cell barcode bead. In step 2710 the isolated cell sample is prepared, including step 2710a of lysis, step 2710b of indexing the spatial barcode, cDNA and target barcode with the cell barcode, and step 2710c of library prep. Due to different amounts of the spatial barcode, cDNA and target barcode molecules/probes in the cell, library prep (and sequencing) may be done separately. For example, indexed spatial barcodes and/or indexed target barcodes may be separated from indexed cDNA by size selection. Alternatively, the original probes in the cell and/or indexing molecules on the bead may incorporate different sequences that allow for selective amplification. Shading of the illustrations of steps 2704 through 2710 indicates these steps are performed in solution. While steps 2706-2710 are adapted from the Cite-Seq workflow and Reap-Seq workflows, and may be run on any of a variety of droplet based platforms. Of note, while the cell barcode sequence hybridizes to poly-A in this example, it may hybridize to any given sequence based on assay design.

### Utility of Spatial Barcoding prior to Cell Dissociation

Sequencing enables highly-plexed detection of DNA, RNA, and even protein targets (e.g., using target barcoded oligonucleotide are conjugated to antibodies that bind the protein targets). Recently, a variety of fluidic platforms including the Fluidigm Cl system, and droplet based platforms have enabled indexing of single cells (such as gDNA or cDNA of single cells) for sequencing. These systems have also been adapted for codetection of protein alongside RNA. However, these highly-plexed single cell assays do not provide spatial resolution, as the cell is dissociated from tissue.

Technologies that enable spatial profiling of gene expression, such as Spatial Transcriptomics and Nanostring nCounter, are not able to achieve single cell resolution with any reasonable throughput. In a Nanostring nCounter like method, oligonucleotide probes are photocleaved by a laser, aspirated, and associated with a barcode that identifies that sampling event. However, the physical need to separate different aspiration events in time and/or space to separately barcode them limits the number of regions that can be analyzed this way. In a Spatial Transcriptomics like approach, RNA from sample is applied to an array of spatially barcoded oligonucleotides, and resolution is limited by the spot size of the array and further by the diffusion of the RNA from the tightly packed cells in the tissue section. In both approaches, the sensitivity and multi-omic assays available to workflows that analyze single cell lysates in a homogenous format are difficult to achieve.

As described herein, associating one or more spatial barcodes with cell in tissue prior to dissociation encodes its spatial location in tissue. This spatial barcode can be indexed with the same cell barcode as is used to index cDNA, gDNA or Antibody and/or bound oligonucleotide sequences in a single cell workflow. The cell barcode can be incorporated alongside the spatial barcode in a reaction product (oligonucleotide sequence) that can be sequenced. For example, an oligonucleotide comprising the cell barcode may hybridize to and extend along an oligonucleotide comprising the spatial barcode, such that the resultant reaction comprises both the cell barcode and the spatial barcode (or specifically its reverse complement). Similarly, the other instances of the same cell barcode sequence may be incorporated with targets on or in the cell, such as cDNA sequences, gDNA sequences, and/or target barcodes sequences (e.g., of an oligonucleotide conjugated to an affinity reagent, such as an antibody).

Further, the combination of spatial barcodes associated with a given cell can pinpoint its location (e.g., within 100 microns, 20 microns, or 10 microns). For the first time, this would allow highly-plexed, transcriptomic or genomic analysis of single cells with spatial resolution, with a potential for multi-omic applications such as protein co-expression. The rich dataset obtained from such an experiment may enable a user to explore the spatial distribution different cell populations and markers in tissue in an interactive way. In addition, machine learning can be applied to identify signatures of cell type distributions that identify a disease, prognosis, or elucidate basic biology.

Embodiments of spatial barcoding described herein include specialized oligonucleotide array and method of use that allows for spatial barcoding of cells before disaggregation of tissue and downstream processing (including single cell isolation and barcoding). This workflow enables nearly any single cell omic or multiomic assay with a sequencing readout to be performed with spatial resolution, and provides higher spatial resolution and cost savings compared to competing techniques. In certain aspects, cost of sequencing (which can be on the order of $1 per cell) can be reduced for the sample by depleting (of all cells or of overabundant cell populations) or conversely enrichment for certain population(s) of interest, such as by fluorescence activated cell sorting (FACS) prior to single cell indexing and sequencing.

Further, the application of the encoding relative spatial proximity of targets in a sample, which is described throughout this application, may be combined with spatial barcoding of dissociated cells, so as to provide high resolution within the cell.

In certain aspects, the tissue (or cellular) sample may be live, and spatial barcoding may be done in real time such that the spatial barcodes associated with a cell record a path it traveled during.

In certain aspects, when gDNA or cDNA is detected, mutations conserved across cells may allow for lineage tracing which can be tied to the location of cells. As such, the spread of cancer cells in a tissue can be traced spatially, and related to lineage and optionally related to other cell characteristics such as protein and/or RNA expression (e.g., via multi-omic approaches enabled for solution based single cell assays).

### Related Technologies and Modifications

Methods and kits described herein are enabled by a variety of technologies described below.

Encoding of relative positions of oligonucleotide probes has been shown by the Zheng et al. in “DNA microscopy: Optics-free spatio-genetic imaging by a stand-alone chemical reaction” (Cell 178.1 (2019): 229-241). However, due to the diffusion of the probes and the reaction products formed by their reaction scheme, the spatial resolution of this approach is limited thus far to cellular resolution at best (on the scale of microns). Specifically, reaction product formed by extension of the overlap primers would be displaced during amplification, and necessarily diffuse. Such reaction products formed by diffusion would obviate any resolution that may be provided by reaction sequences that are immobilized in the sample, as described in embodiments of the subject application.

Formation of reaction products immobilized to a solid surface has been well documented outside of the application of sequence based imaging. Reactions such as bridge amplification for sequencing on lllumina systems allow extension of immobilized oligonucleotides that are proximal and complementary to one another. These reactions can be isothermal due to strain-promoted dehybridization and strand invasion mediated by the concentration of complementary sequences immobilized within reach of one another (e.g., on the scale of nanometers).

Isothermal amplifications via strand displacement may be promoted by used of one or more probes that are hairpins. Hairpin probes may comprise variable sequences and/or barcodes of any probe designs discussed herein, and may include a spaced repeat flanking the variable and/or barcode sequences such that extension along the probe terminates in the same sequence, allowing for successive extensions as described herein. The hairpin probe may have a sequence complementary to the repeats, that competes for hybridization and displaces a reaction product by strand invasion, allowing that reaction product to extend along another probe. Such hairpin design and functionality may be based on the hairpins characterized by Yin et al. in “Immuno-SABER enables highly multiplexed and amplified protein imaging in tissues.” (Nature biotechnology 37.9 (2019): 1080-1090). Such probes may also be used to make long concatemers that provide a series of repeating spatial barcodes, and individual reaction products may be formed by extension along portions of the concatemer (e.g., in the absence of a dNTP which halts extension).

Reaction products may form from extension of immobilized probes, and would therefore themselves be immobilized in (e.g, on or within) the sample. As a reaction product forms, such as by successive extension described herein, it may reach and react with more distant probes (e.g., reaction products formed from more distant probes). Reaction products formed while immobilized to the sample may react with surrounding immobilized probes and/or free probes, in any of a number of reaction schemes described herein and combinations thereof.

As shown in FIG. 7, proteins can cluster on the cell surface within nanometers (e.g., 10-100 nm) from other protein clusters. Target specific oligonucleotide probes and/or reference probes, as described herein, can be bound to the sample in close enough proximity to react with (e.g., hybridize to and extend from) proximal probes also bound to the sample. A nucleotide is roughly 0.3 nm in length, meaning that a sequence 40 nucleotides long would be more than 10 nm long. Oligonucleotide probes may be between 6 and 100 nucleotides long, and may also include a linker that further distances them from a site of attachment to a sample. For example, a PEG5000 linker would provide roughly 50 additional nm in length. As such, two probes bound within 100 nm of one another may be able to reach an hybridize one another. Further, a long oligonucleotide sequence (e.g., greater than 100 nt long, greater than 500 nt long) may be considered a linker provide it is not a template for extension from other probes. Non-nucleotide linkers, such as PEG spacers, are contemplated herein as well.

As discussed herein, resolution may be improved by limiting the diffusion of reaction products. This may be done by immobilizing reaction products covalently, through target specific binding, and/or through the incorporation of a matrix to the sample, such as by gel expansion. Probes may be anchored, or have their diffusion reduced and/or regulated, in a gel expanded sample. Gel expansion workflows are described by Boyden et al. in “Expansion microscopy: principles and uses in biological research.” (Nature methods 16.1 (2019): 33-41).

As longer reaction products are formed, repeats that hybridize to surrounding probes and/or reaction products may allow for phenomenon such as “walking” which may allow the reaction probe to remain bound to the sample at any given time but slowly move across the sample, increasing the range across which it can react while limiting rapid diffusion.

Methods of single cell imaging discussed herein include application of spatial barcodes to a tissue sample, disaggregation, and single cell indexing of spatial barcodes and targets (e.g., protein and/or gene expression) in solution. Disaggregation allows for efficient and multi-omic single cell assays, such as protein and RNA co-detection enabled by assays such as Cite-Seq reported by Smibert et al. in “Simultaneous epitope and transcriptome measurement in single cells.” (Nature methods 14.9 (2017): 865). While Cite-seq and other workflows use the same hybridization sequence (poly-A) for cell indexing of RNA expression as well as for cell indexing of protein expression, I should be understood that other (e.g., target specific) hybridization sequences may be used. As indexed spatial barcodes, protein barcodes, and cDNA may need to be library prepped and sequenced separately, aspects of the subject application include a bead with a cell barcode on sequences that have different 3′ sequences and different primer binding sites to allow for separate library prep and/or amplification.

Modifications of oligonucleotide probes (e.g., spatial barcode probes) to associate with the cell (e.g., cell surface) are provided by a number of established techniques. Modification of oligonucleotides with a lipid group for incorporation into the cell membrane is reported by Gartner et al. in “Efficient targeting of fatty-acid modified oligonucleotides to live cell membranes through stepwise assembly.” (Biomacromolecules 15.12 (2014): 4621-4626). Acrydide functionalized thiol-reactive oligonucleotides can be ordered from IDT, or maleimide functionalized oligonucleotides can be synthesized with a click chemistry (e.g., strain promoted click chemistry, such as DBCO-azide or TCO-tetrazine) intermediate. Such thiol-reactive groups can covalently bind thiol groups presented by the cell, and have favorable kinetics for barcoding cells as shown by Nitz et al. in “Tellurium-based mass cytometry barcode for live and fixed cells.” (Cytometry Part A 93.7 (2018): 685-694). The above reactions are fast (on the order of minutes if not seconds for a suitable number of probes to bind the sample, after which unbound probes can be washed off). The above reactions, or functionalization of the cell surface with an antibody intermediate, may be used to present an oligonucleotide or an avidin (e.g., streptavidin) group on the cell surface without concern for fast kinetics. Hybridization of sample barcode probe to the oligonucleotide, or binding of biotinylated sample barcode probe to streptavidin, may be performed to bind sample barcode probe with fast kinetics. Such methods of rapidly associating sample barcode probe with cells in tissue allow for diffusion limited binding in which the majority of bound probes are within a specific location. The location may be less than 2 mm, less than 1 mm, less than 500 microns, less than 200 microns, less than 100 microns, or less than 50 microns. Additional methods of cell surface functionalization and considerations are generally described by Perriman et al. in “Strategies for cell membrane functionalization.” (Experimental Biology and Medicine 241.10 (2016): 1098-1106). Methods of peptide modifications of oligonucleotides to promote cell uptake are discussed by Juliano et al. in “Covalent conjugation of oligonucleotides with cell-targeting ligands.” (Bioorganic & medicinal chemistry 21.20 (2013): 6217-6223).

The above options for assay design, in combination with the assay schemes described herein, provide myriad sequence based imaging workflows across a number of applications.

We claim:

1. A method of representing the spatial relationships of oligonucleotide probes in a biological sample, the method comprising:
a) contacting a biological sample with a set of oligonucleotide probes; and
b) forming reaction products in the biological sample;
wherein the reaction products are formed between proximal oligonucleotide probes comprising different variable sequences;
wherein a plurality of the oligonucleotide probes are target-specific oligonucleotide probes;
wherein the reaction products that encode the proximity of a plurality of oligonucleotide probes;
wherein the variable sequences of a single oligonucleotide probe are present on a plurality of different reaction products,
wherein one or more of the reaction products comprise a different combination of variable sequences; and
wherein one or more of the reaction products comprise variable sequences from at least 3 different oligonucleotide probes.

2. The method of claim 1, wherein step b) occurs without prior amplification of the target-specific oligonucleotide probes within the sample.

3. The method of claim 2, wherein one or more of the oligonucleotide probes are immobilized during step b).

4. The method of claim 3, wherein one or more of the oligonucleotide probes are bound to a protein target by an antibody intermediate during step b).

5. The method of claim 3, wherein one or more of the oligonucleotides probes are cross-linked to the sample during step b).

6. The method of claim 1, wherein the reaction products encode the proximity of protein targets, of RNA targets, or of protein and RNA targets.

7. The method of claim 1, wherein step b) comprises a first extension of a first oligonucleotide probe along a second oligonucleotide probe to form a first extension product, wherein the first extension product terminates in a 3′ hybridization sequence that then hybridizes to and extends from another oligonucleotide probe or extension product.

8. The method of claim 1, wherein one or more oligonucleotide probes are reference oligonucleotide probes that are distributed non-specifically in the sample.

9. The method of claim 8, wherein the non-specific oligonucleotide probes are functionalized to bind to common functional groups throughout the biological sample

10. The method of claim 1, wherein step b) of forming reaction products is isothermal.

11. The method of claim 1, wherein the reaction products together encode the relative proximity between of at least 20 oligonucleotide probes and/or their targets.

12. The method of claim 1, wherein one or more of the oligonucleotide probes comprise hairpin structures, wherein the hairpin structures allow for isothermal reactions in step b) via strand displacement.

13. The method of claim 1, wherein each of a plurality of the target-specific oligonucleotide probes comprise a target barcode sequence encoding the specific target.

14. The method of claim 1, further comprising step c) of sequencing the reaction products, wherein the co-occurrence of variable sequences in reaction products indicates proximity of oligonucleotide probes in the sample that comprised the co-occurring variable sequences.

15. The method of claim 14, further comprising step d) of visually representing the relative proximity of oligonucleotide probes and/or their targets based on the sequences of their reaction products.

16. The method of claim 1, wherein the sample is expanded by gel expansion, and wherein one or more oligonucleotide probes are reference oligonucleotide probes that are distributed non-specifically in the sample, and wherein reference oligonucleotides are bound by covalent binding to the gel matrix.

17. The method of claim 1, wherein one or more oligonucleotide probes comprise a spatial barcode, wherein probes with the spatial barcode are spatially organized before application to the sample such that spatial barcodes are related to known locations.

18. The method of claim 1, wherein some but not all of the oligonucleotide probes incorporated into the reaction product are unbound.

19. The method of claim 1, wherein the majority of oligonucleotide probes encoded in a reaction product of the plurality of reaction products are within 100 nm of the majority of other oligonucleotide probes in the same reaction product.

20. A kit for encoding spatial relationships of targets in a biological sample, the kit comprising: a set of oligonucleotide probes, wherein one or more of the probes in the set are target specific oligonucleotide probes; wherein one or more oligonucleotide probes in the kit comprise a first complimentary sequence at the 3′ end that hybridizes to other oligonucleotide probes in the set; and wherein extending from the first hybridization sequence encodes one or more identifiers of the template oligonucleotide probe and terminates in a second hybridization sequence that can hybridize to, and extend along, another oligonucleotide probe in the set.
